(19) United States
(12) Patent Application Publication
(10) Pub. No.: US 2024/0150768 A1
(43) Pub. Date: May 9, 2024

(54) METHODS FOR THE TREATMENT OF NASH WITH ADVANCED FIBROSIS AND/OR CIRRHOSIS

(71) Applicant: Boehringer Ingelheim International GmbH, Ingelheim am Rhein (DE)

(72) Inventors: Ingo UPHUES, Biberach an der Riss (DE); Marc ABRAMS, Natick, MA (US); Andre BROERMANN, Schemmerhofen (DE); Bob Dale BROWN, Littleton, MA (US); Kevin CRAIG, Wilmington, MA (US); Henryk T. DUDEK, Belmont, MA (US); Boris FERGER, Biberach an der Riss (DE); Martin Lee KOSER, Westford, MA (US); Jihye PARK, Cambridge, MA (US); Utsav SAXENA, Watertown, MA (US)

(21) Appl. No.: 18/483,540

(22) Filed: Oct. 10, 2023

Related U.S. Application Data
(60) Provisional application No. 63/379,030, filed on Oct. 11, 2022.

(51) Int. Cl. C12N 15/113; A61P 1/16

(52) U.S. Cl. CPC C12N 15/1137; A61P 1/16; C12Y 207/01003; C12N 2310/14; C12N 2320/32; C12N 2320/35

(57) ABSTRACT

The present invention provides methods for the treatment of non-alcoholic steatohepatitis (NASH) including advanced fibrotic and/or cirrhotic stages of NASH. The methods include administration of a compound comprising a double stranded RNAi (dsRNAi) oligonucleotide, or a pharmaceutically acceptable salt thereof, to the patient. The dsRNAi oligonucleotide optionally contains an antisense strand and a sense strand, wherein the antisense strand and the sense strand form a duplex region, and wherein the antisense strand comprises a region of complementarity to a KHK mRNA target sequence.

### RELATED APPLICATION DISCLOSURE

This application is a nonprovisional of U.S. Provisional Application No. U.S. 63/379,030, pending, filed Oct. 11, 2022, which is hereby incorporated by reference herein in its entirety.

### SEQUENCE DISCLOSURE

This application includes, as part of its disclosure, a “Sequence Listing XML” pursuant to 37 C.F.R. § 1.831(a) which is submitted in XML file format via the USPTO patent electronic filing system in a file named “01-3539-US-2_ST.26_SL_2023-09-28.xml”, created on Sep. 28, 2023, and having a size of 173,000 bytes, which is hereby incorporated by reference herein in its entirety.

### FIELD OF THE INVENTION

This invention relates to double stranded (ds) RNAi oligonucleotides targeting ketohexokinase (KHK) mRNA for use in methods for the treatment of the advanced fibrotic and/or cirrhotic stages of non-alcoholic steatohepatitis (NASH), optionally in combination with other pharmaceutically active substances. In addition, the invention relates to pharmaceutical compositions comprising said dsRNAi oligonucleotides and optionally other pharmaceutically active substances and to methods for the treatment of the advanced fibrotic and/or cirrhotic stages of NASH with said dsRNAi oligonucleotides or compositions, optionally in combination with other pharmaceutically active substances.

### BACKGROUND OF THE INVENTION

Non-alcoholic fatty liver disease (NAFLD), the hepatic component of metabolic syndrome, is the most common chronic liver disease, with an incidence rate of 30% in the United States, Europe, and Japan. Roughly 10 to 12% of patients with NAFLD have NASH, consisting of liver steatosis, inflammation, and progressive hepatocyte injury, which may result in fibrosis. The staging of fibrosis (F1-F4) represents a qualitative descriptor of disease progression: While stages F1 to F3 describe the initial, intermediate, and advanced stages of fibrosis, stage F4 refers to the cirrhotic stage of NASH. The cirrhotic stage (F4) is further classified into two stages: compensated and decompensated, with clinical decompensation being defined by the development of ascites, variceal hemorrhage, encephalopathy, and jaundice. With the rising prevalence of obesity, diabetes mellitus, and subsequently NASH, an accompanying increase in advanced stages of fibrosis/cirrhosis (in particular NASH F4) is expected. Hence, there will be an increasing need for targeted therapies with the primary objective of preventing cirrhosis-related decompensation.

To date, no treatment has been approved for patients with NASH, let alone with advanced fibrosis and/or cirrhosis or, in particular, NASH F4. Standard of care for patients with NASH F4 compensated-cirrhosis is focused on lifestyle modifications (diet/exercise), treatment of the respective underlying metabolic diseases (e.g., antihypertensive, antihyperglycemic, or antihyperlipidemic treatments), as well as symptomatic treatment such as off-label use of beta-blockers to prevent decompensation and symptomatic treatment of decompensation events (e.g., endoscopic varices ligation, diuretics for ascites). Thus, there is a high medical need for effective treatments of NASH.

Dietary components may contribute to NAFLD/NASH and liver fibrosis. Fructose, either from table sugar (saccharose) or from high fructose corn sirup (HFCS), is an important component of a variety of food products, e.g., soft drinks, sweets, or even flavoured yogurt. While the worldwide total fructose consumption is stable over the last years, the HFCS consumption has almost doubled since 1970.

High concentrations of fructose can readily lead to induction of fructose metabolism in liver via fructose-1-phosphate (F-1-P). The phosphorylation of fructose is catalysed by the enzyme ketohexokinase (KHK), an important enzyme in fructose metabolism. Excessive fructose phosphorylation is associated with adenosine triphosphate (ATP) depletion in hepatocytes, leading to hepatocyte death, consecutive inflammation, and subsequently increased fibrosis in the liver. F-1-P further enters metabolic pathways towards triglyceride synthesis (de novo lipogenesis). Knockdown of KHK protein expression in the liver with small interfering ribonucleic acid (siRNA) will prevent the metabolism of fructose towards F-1-P. This siRNA action will result in decreased de novo lipogenesis and reduced ATP depletion, subsequently leading to prevention of hepatocyte death and liver fibrosis and is a therapeutic mechanism which may help patients with NASH.

Dicer-substrate siRNA oligonucleotides designed to target KHK messenger ribonucleic acid (mRNA) thereby reducing levels of KHK protein in the liver are known from the prior art, e.g., from WO 2015/123264, WO 2020/060986, WO 2021/178736, WO 2022/182574, and WO 2022/218941.

### BRIEF DESCRIPTION OF THE DRAWINGS

FIG. 1: Absolute Body weight (g) from male C57/BL6 mice during the study. Mice were subcutaneously injected at day 0 and day 3 with 6 mg/kg and with 3 mg/kg at day 7, 14, 21, 28, 35, 42, 49, 56, 63, 70, 77 of a Compound A or vehicle. Values are expressed as mean of n=6-14+SEM.

FIG. 2: Absolute liver weight (g) from male C57/BL6 mice at the end of the study. Mice were treated as outlined for FIG. 1. Values are expressed as mean of n=6-14+SEM. Dunnett's test one-factor linear model. ***: P<0.001 compared to DIO-NASH Vehicle.

FIG. 3: Liver sinusoidal fibrosis (Picro Sirius Red (PSR) staining % fractional area) from male C57/BL6 mice at the end of the study. Mice were treated as outlined for FIG. 1. Values are expressed as mean of n=6-14+SEM. Dunnett's test one-factor linear model. *: P<0.05, **: P<0.01, ***: P<0.001 compared to DIO-NASH Vehicle.

FIG. 4: Liver periportal fibrosis (Picro Sirius Red (PSR) staining % fractional area) from male C57/BL6 mice at the end of the study. Mice were treated as outlined for FIG. 1. Values are expressed as mean of n=6-14+SEM. Dunnett's test one-factor linear model. *: P<0.05, **: P<0.01, ***: P<0.001 compared to DIO-NASH Vehicle.

FIG. 5: Liver Collagen 1a1 (% fractional area) from male C57/BL6 mice at the end of the study. Mice were treated as outlined for FIG. 1. Values are expressed as mean of n=6-14+SEM. Dunnett's test one-factor linear model. *: P<0.05, **: P<0.01, ***: P<0.001 compared to DIO-NASH Vehicle.

FIG. 6: Relative and total liver lipid (% fractional area and absolute values in mg) from male C57/BL6 mice at the end of the study. Mice were treated as outlined for FIG. 1. Values are expressed as mean of n=6-14+SEM. Dunnett's test one-factor linear model. *: P<0.05, **: P<0.01, ***: P<0.001 compared to DIO-NASH Vehicle.

FIGS. 7A-7C: Percent (%) KHK mRNA remaining in liver biopsies from non-human primates (NHP) 28 days (FIG. 7A), 56 days (FIG. 7B), and 84 days (FIG. 7C) after a single dose of specified GalNAc-constructs. NHP were subcutaneously injected with 6 mg/kg of GalNAc-KHK on Study Day 0. The percent indicated is the average reduction in KHK-mRNA compared to a PBS control.

FIG. 7D: Changes in KHK mRNA in liver biopsies taken at various time points from NHP (as treated in FIGS. 7A-7C) after a single dose of GalNAc-KHK constructs.

FIGS. 8A-8C: Percent (%) KHK protein remaining in liver biopsies from non-human primates (NHP) 28 days (FIG. 8A), 56 days (FIG. 8B), and 84 days (FIG. 8C) after treatment. NHP were treated as in FIGS. 7A-7C. The percent indicated is the average reduction in KHK-protein compared to a PBS control.

FIG. 8D: Changes in KHK protein in liver biopsies taken at various time points from NHP (as treated in FIGS. 7A-7C) after a single dose of GalNAc-KHK constructs.

FIGS. 9A-9C: Correlation between remaining KHK mRNA expression and remaining KHK protein expression in liver biopsies from NHP treated with a single dose of GalNAc-KHK constructs. Correlation among all constructs is compared at days 28 (FIG. 9A), 56 (FIG. 9B), and 84 (FIG. 9C) after dosing. Individual dots represent individual biopsies.

FIG. 10A: Percent (%) KHK mRNA remaining in liver biopsies from male C57/BL6 mice at the end of the study. Mice were treated as outlined for FIG. 1. Values are expressed as mean of n=6-14+SEM compared to DIO-NASH vehicle.

FIG. 10B: Percent (%) KHK protein remaining in liver biopsies from male C57/BL6 mice at the end of the study. Mice were treated as outlined for FIG. 1. Values are expressed as mean of n=6-14+SEM compared to DIO-NASH vehicle.

### SUMMARY OF THE INVENTION

In a first aspect, the present invention relates to a double stranded RNAi (dsRNAi) oligonucleotide for reducing ketohexokinase (KHK) expression, or a pharmaceutically acceptable salt thereof, for use in a method for the treatment of the advanced fibrotic and/or cirrhotic stages of non-alcoholic steatohepatitis (NASH) in a patient in need thereof.

In a second aspect, the present invention relates to a pharmaceutical composition comprising one or more of said dsRNAi oligonucleotides, or one or more pharmaceutically acceptable salts thereof, and one or more pharmaceutically acceptable excipients for use in a method for the treatment of the advanced fibrotic and/or cirrhotic stages of NASH in a patient in need thereof.

Likewise, the present invention relates to a method for the treatment of the advanced fibrotic and/or cirrhotic stages of NASH in a patient in need thereof, the method being characterized in that one or more of said dsRNAi oligonucleotides, or one or more pharmaceutically acceptable salts thereof, and/or one or more of said pharmaceutical compositions is administered to the patient.

Likewise, the present invention relates to the use of one or more of said dsRNAi oligonucleotides, or one or more pharmaceutically acceptable salt thereof, in the manufacture of a medicament for the treatment of the advanced fibrotic and/or cirrhotic stages of NASH in a patient in need thereof.

Further aspects of the present invention will become apparent to the person skilled in the art directly from the foregoing and following description.

### General Terms and Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

“Administer”, “administering”, “administration” and the like refers to providing a substance to a subject in a manner that is pharmacologically useful (e.g., to treat a condition in the subject).

The terms “combination” or “combined” within the meaning of this invention may include, without being limited, fixed and non-fixed (e.g., free) forms (including kits) and uses, such as, e.g., the simultaneous, sequential, or separate use of the components or ingredients.

“Complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In particular, complementary nucleotides can base pair in the Watson-Crick manner or in any other manner that allows for the formation of stable duplexes. Also, two nucleic acids may have regions of multiple nucleotides that are complementary with each other to form regions of complementarity, as described herein.

“Deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

All the “doses” or dosage units of a physiologically acceptable salt of one of the above-mentioned active compounds should be understood as being doses or dosages of the active compound itself.

“Double-stranded oligonucleotide” or “ds oligonucleotide” refers to an oligonucleotide that is substantially in a duplex form. The complementary base-pairing of duplex region(s) of a double-stranded oligonucleotide may be formed between antiparallel sequences of nucleotides of covalently separate nucleic acid strands. Also, complementary base-pairing of duplex region(s) of a double-stranded oligonucleotide may be formed from a single nucleic acid strand that is folded (e.g., via a hairpin) to provide complementary antiparallel sequences of nucleotides that base pair together. In particular, a double-stranded oligonucleotide comprises two covalently separate nucleic acid strands that are partially duplexed (e.g., having overhangs at one or both ends). Also, a double-stranded oligonucleotide may comprise antiparallel sequence of nucleotides that are partially complementary, and thus, may have one or more mismatches, which may include internal mismatches or end mismatches.

“Duplex,” in reference to nucleic acids (e.g., oligonucleotides), refers to a structure formed through complementary base pairing of two antiparallel sequences of nucleotides.

“Excipient” refers to a non-therapeutic agent that may be included in a composition, for example, to provide or contribute to a desired consistency or stabilizing effect.

“Hepatocyte” refers to a cell of the parenchymal tissues of the liver. These cells make up about 70%-85% of the liver's mass and manufacture serum albumin, FBN and the prothrombin group of clotting factors (except for Factors 3 and 4).

The term “ketohexokinase” (“KHK”) refers to an enzyme, specifically a hepatic fructokinase, that catalyzes the phosphorylation of fructose. The KHK gene encodes two protein isoforms (KHK-A and KHK-C). The two products are generated from the same primary transcript by alternative splicing. The term “KHK” is intended to refer to both isoforms unless stated otherwise. “KHK” may also refer to the gene which encodes the protein.

“Liver fibrosis” or “fibrosis of the liver” refers to an excessive accumulation in the liver of extracellular matrix proteins, which could include collagens (I, III, and IV), FBN, undulin, elastin, laminin, hyaluronan and proteoglycans resulting from inflammation and liver cell death. Liver fibrosis, if left untreated, may progress to cirrhosis, liver failure or liver cancer.

“Loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cell), the two antiparallel regions, which flank the unpaired region, hybridize to form a duplex (referred to as a “stem”).

“Modified internucleotide linkage” refers to an internucleotide linkage having one or more chemical modifications when compared with a reference internucleotide linkage comprising a phosphodiester bond. A modified nucleotide may be a non-naturally occurring linkage. Typically, a modified internucleotide linkage confers one or more desirable properties to a nucleic acid in which the modified internucleotide linkage is present. For example, a modified internucleotide linkage may improve thermal stability, resistance to degradation, nuclease resistance, solubility, bioavailability, bioactivity, reduced immunogenicity, etc.

“Modified nucleotide” refers to a nucleotide having one or more chemical modifications when compared with a corresponding reference ribonucleotide (A, C, G, T, U). A modified nucleotide may be a non-naturally occurring nucleotide. A modified nucleotide may have one or more chemical modification in its sugar, nucleobase and/or phosphate group. Also, a modified nucleotide may have one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

“Nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

“Oligonucleotide” refers to a short nucleic acid (e.g., less than about 100 nucleotides in length). An oligonucleotide may be single-stranded (ss) or ds. An oligonucleotide may or may not have duplex regions. For instance, an oligonucleotide may be, but is not limited to, a small interfering RNA (siRNA) or a Dicer substrate interfering RNA (DsiRNA). A double-stranded RNA oligonucleotide (dsRNA) may be an RNAi oligonucleotide.

“Overhang” refers to one or more terminal non-base pairing nucleotides resulting from one strand or region extending beyond the terminus of a complementary strand with which the one strand or region forms a duplex. An overhang may comprise one or more unpaired nucleotides extending from a duplex region at the 5′ terminus or 3′ terminus of a dsRNA, for instance, the overhang may be a 3′ or 5′ overhang on the antisense strand or sense strand of a dsRNA.

When this invention refers to “patients” in need of treatment, it relates primarily to treatment in humans.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

“Pharmaceutically acceptable salt” refers to derivatives of the disclosed compounds wherein the parent compound is modified by making organic or inorganic acid or base salts thereof. Examples of pharmaceutically acceptable salts include, but are not limited to, mineral or organic acid salts of basic residues such as amines; alkali or organic salts of acidic residues such as carboxylic acids; and the like.

Salts of other acids than those mentioned above which for example are useful for purifying or isolating the compounds of the present invention (e.g., trifluoro acetate salts) also comprise a part of the invention.

“Phosphate analog” refers to a chemical moiety that mimics the electrostatic and/or steric properties of a phosphate group. A phosphate analog may be positioned at the 5′ terminal nucleotide of an oligonucleotide in place of a 5′-phosphate, which is often susceptible to enzymatic removal. Thus, a 5′ phosphate analog may contain a phosphatase-resistant linkage. Examples of phosphate analogs include, but are not limited to, 5′ phosphonates, such as 5′ methylene phosphonate (5′-MP) and 5′-(E)-vinylphosphonate (5′-VP). Also, an oligonucleotide may have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., US Patent Publication No. 2019-0177729. Other modifications have been developed for the 5′ end of oligonucleotides (see, e.g., Intl. Patent Application No. WO 2011/133871; U.S. Pat. No. 8,927,513; and Prakash et al. (2015) NUCLEIC ACIDS RES. 43:2993-3011).

“Reduced expression” of a gene (e.g., KHK) refers to a decrease in the amount or level of RNA transcript (e.g., KHK mRNA) or protein encoded by the gene and/or a decrease in the amount or level of activity of the gene in a cell, a population of cells, a sample, or a subject, when compared to an appropriate reference (e.g., a reference cell, population of cells, sample or subject). For example, the act of contacting a cell with an oligonucleotide herein (e.g., an oligonucleotide comprising an antisense strand having a nucleotide sequence that is complementary to a nucleotide sequence comprising KHK mRNA) may result in a decrease in the amount or level of KHK mRNA, protein and/or activity (e.g., via degradation of KHK mRNA by the RNAi pathway) when compared to a cell that is not treated with the dsRNA. Similarly, and as used herein, “reducing expression” refers to an act that results in reduced expression of a gene (e.g., KHK). “Reduction of KHK expression” refers to a decrease in the amount or level of KHK mRNA, KHK protein and/or KHK activity in a cell, a population of cells, a sample or a subject when compared to an appropriate reference (e.g., a reference cell, population of cells, sample, or subject).

“Region of complementarity” refers to a sequence of nucleotides of a nucleic acid (e.g., a dsRNA) that is sufficiently complementary to an antiparallel sequence of nucleotides to permit hybridization between the two sequences of nucleotides under appropriate hybridization conditions (e.g., in a phosphate buffer, in a cell, etc.). An oligonucleotide herein may comprise a targeting sequence having a region of complementarity to a mRNA target sequence. In particular, the region of complementarity may be full complementary. Also, the region of complementarity may be partially complementary (e.g., up to 3 nucleotide mismatches).

“Ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

“dsRNAi oligonucleotide” refers to a double-stranded oligonucleotide having a sense strand (passenger) and antisense strand (guide), in which the antisense strand or part of the antisense strand is used by the Argonaute 2 (Ago2) endonuclease in the cleavage of a target mRNA; e.g., dsRNAi oligonucleotides that target KHK mRNA and reduce KHK expression are referred to herein as KHK-targeting dsRNAi oligonucleotides.

“Strand” refers to a single, contiguous sequence of nucleotides linked together through internucleotide linkages (e.g., phosphodiester linkages or phosphorothioate linkages). A strand may have two free ends (e.g., a 5′ end and a 3′ end).

“Targeting ligand” refers to a molecule (e.g., a carbohydrate, amino sugar, cholesterol, polypeptide, or lipid) that selectively binds to a cognate molecule (e.g., a receptor like asialoglycoprotein receptor (ASGPR)) of a tissue or cell of interest and that is conjugatable to another substance for purposes of targeting the other substance to the tissue or cell of interest. For example, a targeting ligand may be conjugated to an oligonucleotide for purposes of targeting the oligonucleotide to a specific tissue or cell of interest. In particular, a targeting ligand selectively binds to a cell surface receptor. Accordingly, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. A targeting ligand may be conjugated to an oligonucleotide via a linker that is cleaved following or during cellular internalization such that the oligonucleotide is released from the targeting ligand in the cell.

“Tetraloop” refers to a loop that increases stability of an adjacent duplex formed by hybridization of flanking sequences of nucleotides. The increase in stability is detectable as an increase in melting temperature (Tm) of an adjacent stem duplex that is higher than the Tm of the adjacent stem duplex expected, on average, from a set of loops of comparable length consisting of randomly selected sequences of nucleotides. Examples of tetraloops include the UNCG family of tetraloops (e.g., UUCG), the GNRA family of tetraloops (e.g., GAAA), and the CUUG tetraloop.

The term “therapeutically effective amount” means an amount of a compound of the present invention that (i) treats or prevents the particular disease or condition, (ii) attenuates, ameliorates, or eliminates one or more symptoms of the particular disease or condition, or (iii) prevents or delays the onset of one or more symptoms of the particular disease or condition described herein.

The terms “treatment” and “treating” as used herein embrace both therapeutic, i.e. curative and/or palliative, especially abortive and/or acute, treatment and preventative, i.e. prophylactic, treatment.

Therapeutic treatment (“therapy”) refers to the treatment of patients having already developed one or more of said conditions in manifest, acute or chronic form. Therapeutic treatment may be symptomatic treatment in order to relieve the symptoms of the specific indication or causal treatment in order to reverse or partially reverse the conditions of the indication or to stop or slow down progression of the disease.

Preventative treatment (“prevention”, “prophylaxis”) refers to the treatment of patients at risk of developing one or more of said conditions, prior to the clinical onset of the disease in order to reduce said risk.

The terms “treatment” and “treating” include the administration of one or more active compounds, in particular therapeutically effective amounts thereof, in order to prevent or delay the onset of the symptoms or complications and to prevent or delay the development of the disease, condition or disorder and/or in order to eliminate or control the disease, condition or disorder as well as to alleviate the symptoms or complications associated with the disease, condition or disorder.

### DETAILED DESCRIPTION OF THE INVENTION

The present invention allows for an efficient treatment of the advanced fibrotic and/or cirrhotic stages of NASH, in particular NASH F4, in patients by administration of dsRNAi oligonucleotides targeting KHK mRNA.

In a first aspect, the present invention relates to a double stranded RNAi (dsRNAi) oligonucleotide for reducing ketohexokinase (KHK) expression, or a pharmaceutically acceptable salt thereof, for use in a method for the treatment of the advanced fibrotic and/or cirrhotic stages of non-alcoholic steatohepatitis (NASH) in a patient in need thereof (e.g., stage F3 of NASH and/or stage F4 of NASH).

As liver fibrosis is the major determinant of this stage of the disease, the reduction of liver fibrosis is expected to have strong therapeutic impact for the treatment of cirrhotic stages of NASH.

It has surprisingly been found in a mouse model for diet-induced liver fibrosis that a reduction of KHK expression in the liver by the administration of a dsRNAi oligonucleotide results in antifibrotic effects (see Example 2).

It has surprisingly been found in a mouse model for diet-induced liver fibrosis that a reduction of KHK expression in the liver by the administration of a dsRNAi oligonucleotide results in antifibrotic effects without reduction of liver lipids (see Example 2).

It has surprisingly been found in a mouse model for diet-induced liver fibrosis that a reduction of KHK expression in the liver by the administration of a dsRNAi oligonucleotide results in antifibrotic effects and it is not associated with significant body or liver weight loss (see Example 2).

It has surprisingly been found in a mouse model for diet-induced liver fibrosis that a reduction of KHK expression in the liver by the administration of a dsRNAi oligonucleotide results in antifibrotic effects without reduction of liver lipids and it is not associated with significant body or liver weight loss (see Example 2).

While it had been described before that knockdown of KHK in the liver (siRNA-mediate) may improve hepatic steatosis (Softic et al., J Clin Invest. 2017; 127(11): 4059-4074), which is in line with theoretical considerations on the metabolic role of KHK, the same study had concluded that “The severity of other NASH features, e.g., [. . . ] fibrosis, was minimal in all groups of mice at 10 weeks on diet and was not affected by KHK siRNA administration.” Thus, the observation of antifibrotic efficacy of dsRNAi agents targeting KHK in the liver without reduction of liver lipids could not have been predicted based on the teachings of the prior art.

In addition, it had been described before that inhibition of KHK enzyme activity using PF-06835919 in human liver tissue reduces triglyceride (TG) accumulation (Shepherd at al., JHEP Reports, 2021, 3(2): 100217, page 6). The same study has concluded that “Inhibition of ketohexokinase enzyme activity halts this conversion, reducing TG accumulation, and cellular stress and causes a consequential reduction in fibrogenesis”. However, no reduction in fibrogenesis by inhibiting KHK enzyme activity was demonstrated by Shepherd using the small molecule PF-06835919, even less using an siRNA.

Consequently, it was unexpected that dsRNAi oligonucleotides targeting KHK in the liver are particularly suitable for the therapy and/or prevention of the advanced fibrotic and/or cirrhotic stages of NASH.

Thus, as a result of the investigations underlying the present invention, dsRNAi oligonucleotides that are able to knock-down KHK mRNA and to reduce the amount of KHK mRNA and/or KHK protein in the human liver are considered to be particularly suitable for the therapy and/or prevention of the advanced fibrotic and/or cirrhotic stages of NASH in human patients.

Due to the observed antifibrotic effects of dsRNAi oligonucleotides effectively targeting human KHK in the liver, beneficial effects of administering such agents may be expected especially for advanced fibrotic stages of NASH (e.g., NASH F3), in particular for the different cirrhotic stages of NASH (e.g., compensated or decompensated NASH F4).

According to one embodiment, the patient is a human patient.

According to another embodiment, the dsRNAi oligonucleotide is for reducing human KHK expression, preferably in the liver.

According to another embodiment, the dsRNAi oligonucleotide is for reducing human KHK-C expression, preferably in the liver.

According to one embodiment, the method is for the treatment of initial fibrotic stages of NASH (e.g., NASH F1).

According to one embodiment, the method is for the treatment of intermediate fibrotic stages of NASH(e.g., NASH F2).

According to one embodiment, the method is for the treatment of advanced fibrotic stages of NASH.

According to another embodiment, the method is for the treatment of the compensated and/or decompensated cirrhotic stage of NASH.

According to another embodiment, the method is for the treatment of the compensated cirrhotic stage of NASH.

According to another embodiment, the method is for the treatment of the decompensated cirrhotic stage of NASH.

According to another embodiment, the method is for the treatment of NASH F3.

According to another embodiment, the method is for the treatment of compensated and/or decompensated NASH F4.

Also, different from what had been suggested by the findings of Softic et al., it has surprisingly been observed in the above-mentioned mouse model that a reduction of KHK expression in the liver by the administration of a dsRNAi oligonucleotide is not associated with significant body or liver weight loss (see Example 2).

Thus, dsRNAi oligonucleotides described herein may be particularly suitable for the treatment of patients who do not benefit from weight loss, in particular for the treatment of patients for whom weight loss, e.g., caused by medical treatment, is undesired. For instance, weight loss may be undesired for patients suffering from advanced fibrotic and/or cirrhotic stages of NASH, in particular from NASH F4, or for patients that are underweight for different reasons.

Also, dsRNAi oligonucleotides described herein may be particularly suitable for combination treatments with therapeutic agents that may cause or do cause a loss of body weight and/or liver weight, e.g., agents for the treatment of non-alcoholic fatty liver disease (NAFLD), NASH, diabetes, obesity, metabolic syndrome, dyslipidemia, hypercholesterolemia, hypertension, cardiovascular diseases, and the like. Agents that may cause or do cause a loss of body weight and/or liver weight are known to the one skilled in the art and include, but are not limited to, agents that are intended to cause weight loss, e.g., agents for the treatment of obesity like orlistat, phentermine-topiramate, naltrexone-bupropion, liraglutide, semaglutide, tirzepatide, agonists of the neuropeptide Y receptor Y2 (NPY2R), of the glucagon receptor (GCGR), of the glucagon-like peptide-1 receptor (GLP-1R), agonists of GCGR/GLP-1R and of NPY2R/GCGR/GLP-1R, agonists of the neuromedin U receptor 2 (NMUR2), agonists of the fibroblast growth factor 21 receptor (FGF21R), and agonists of GLP-1R/FGF21R, as well as agents that may cause weight loss as a side effect, in particular approved agents for which weight loss is mentioned as a potential side effect in the drug label or has been described in the scientific literature (e.g., Domecq et al., J Clin Endocrinol Metab. 2015, 100(2), 363-370).

For the treatment of advanced fibrotic and/or cirrhotic stages of NASH described herein, one or more dsRNAi oligonucleotides are administered to a subject having advanced fibrotic and/or cirrhotic stages of NASH (e.g., NASH F4) such that KHK expression in the liver is reduced in the subject, thereby treating the subject. The dsRNAi oligonucleotides are used and/or administered alone or in combination (concurrently, sequentially, or intermittently) with other agents suitable for the treatment of NAFLD and/or NASH, including, but not limited to, agents that may cause or do cause a loss of body weight and/or improvements in liver steatosis and/or liver function. The dsRNAi oligonucleotides may also be used and/or administered in combination (concurrently, sequentially, or intermittently) with agents suitable for the treatment of diseases like diabetes, obesity, metabolic syndrome, dyslipidemia, hypercholesterolemia, hypertension, and/or cardiovascular diseases. Agents suitable for the treatment and/or combination treatment of the above-mentioned diseases are known to the one skilled in the art and include, but are not limited to, agents that have achieved regulatory approval or have entered clinical trials.

According to one embodiment, the patient is a human patient who does not benefit from weight loss.

According to another embodiment, the patient is a human patient for whom weight loss is undesired.

According to one embodiment, the dsRNAi oligonucleotide is administered alone.

According to another embodiment, the dsRNAi oligonucleotide is administered in combination with at least one further agent for the treatment of NAFLD and/or NASH.

According to another embodiment, the dsRNAi oligonucleotide is administered in combination with at least one further agent for the treatment of diabetes, obesity, metabolic syndrome, dyslipidemia, hypercholesterolemia, hypertension, and/or cardiovascular diseases.

According to another embodiment, the dsRNAi oligonucleotide is administered in combination with at least one further agent that causes a loss of body weight and/or liver weight, e.g., agents for the treatment of NAFLD, NASH, diabetes, obesity, metabolic syndrome, dyslipidemia, hypercholesterolemia, hypertension, and/or cardiovascular diseases.

According to another embodiment, the treatment of advanced fibrotic and/or cirrhotic stages of NASH is a monotherapy of the dsRNAi oligonucleotide.

According to another embodiment, the treatment of advanced fibrotic and/or cirrhotic stages of NASH is a combination treatment of the dsRNAi oligonucleotide(s) with at least one further agent for the treatment of NAFLD and/or NASH.

According to another embodiment, the treatment of advanced fibrotic and/or cirrhotic stages of NASH is a combination treatment of the dsRNAi oligonucleotide(s) with at least one further agent for the treatment of NAFLD and/or NASH that cause a loss of body weight and/or liver weight.

The appropriate dosage regimen for any one subject may depend on certain factors, including the subject's size, body surface area, age, the particular composition to be administered, the active ingredient(s) in the composition, time and route of administration, general health, and other drugs being administered concurrently.

In particular, the specific dose and the frequency of said administration to achieve sufficient reduction of KHK expression in the liver will depend on the potency and duration of action of the specific dsRNAi oligonucleotide, but will be chosen such that the amount of KHK mRNA and/or KHK protein in the liver is reduced sufficiently to produce beneficial effects on the progression of advanced fibrotic and/or cirrhotic stages of NASH (e.g., NASH F4), either in terms of therapy or prevention. For instance, the choice of dose and frequency of administration results in a reduction of the amount of KHK mRNA and/or KHK protein in the subject's liver of at least about 70%, preferably at least about 80% or 85%, more preferably at least about 90% or 95%. Said reduction of the amount of KHK mRNA and/or KHK protein may be determined by comparison with the amount of KHK mRNA and/or KHK protein in a reference or control subject, i.e., a subject not receiving the dsRNAi oligonucleotide(s) or receiving one or more control dsRNAi oligonucleotides, or—preferably—by comparison with the amount of KHK mRNA and/or KHK protein prior to administration of the dsRNAi oligonucleotide(s). Said amount or level of KHK mRNA and/or KHK protein may be determined, e.g., from liver biopsy samples from the subject.

According to one embodiment, the dsRNA oligonucleotide for reducing KHK expression is administered subcutaneously.

According to another embodiment, the dsRNAi oligonucleotide is administered at a dose in the range from about 0.01 mg to about 10 mg per kg body weight, more specifically in the range from about 0.1 mg to about 6 mg per kg body weight, preferably in a dose below 1 mg per kg body weight.

According to another embodiment, the dsRNAi oligonucleotide is administered at a dose in the range from about 1 mg to about 1000 mg, more specifically in the range from about 10 mg to about 700 mg, preferably at a dose below 100 mg.

According to another embodiment, the dsRNAi oligonucleotide is administered on a regular basis, e.g., in intervals of 1, 2, 3, 4, 5, or 6 months, preferably in intervals of 1, 2, or 3 months.

According to a preferred embodiment, the dsRNAi oligonucleotide is administered subcutaneously in intervals of 1, 2, or 3 months at a dose below 100 mg (e.g., at a dose below 1 mg per kg body weight).

According to another embodiment, the choice of dose and frequency of administration results in a reduction of the amount of KHK mRNA and/or KHK protein, preferably in a reduction of the amount of KHK-C protein, in the subject's liver of at least about 70%, preferably at least about 80% or 85%, more preferably at least about 90% or 95%.

dsRNAi oligonucleotides that are able to knock-down KHK mRNA in vitro and/or in vivo and may hence reduce the amount of KHK mRNA and/or KHK protein in the human liver are described in the prior art and/or herein. Human KHK mRNA sequences and preferred target sequences as well as dsRNAi oligonucleotides targeting human KHK mRNA are disclosed, for instance, in WO 2015/123264, WO 2020/060986, WO 2021/178736, WO 2022/182574, and U.S. Ser. No. 17/717,174 the entire contents of which are incorporated herein by reference.

The dsRNAi oligonucleotide for reducing KHK expression comprises an antisense strand and a sense strand, wherein the antisense strand and the sense strand form a duplex region, and wherein the antisense strand comprises a region of complementarity to a KHK mRNA target sequence.

According to one embodiment, said sense strand and said antisense strand are separate strands, i.e., they are not covalently linked.

According to another embodiment, said sense strand is 19 to 36 nucleotides in length, more preferably 19-21 (i.e., 19, 20, or 21) or 36 nucleotides in length.

According to another embodiment, said antisense strand is 19 to 23 nucleotides in length, i.e., 19, 20, 21, 22, or 23 nucleotides in length.

According to another embodiment, said region of complementarity is fully complementary to the KHK mRNA target sequence.

According to another embodiment, said region of complementarity is at least 19 nucleotides in length, e.g., 19-22, i.e., 19, 20, 21, or 22 nucleotides in length.

According to another embodiment, said duplex region is at least 19 nucleotides in length, e.g., 19-21, i.e., 19, 20, or 21 nucleotides in length.

According to another embodiment, said dsRNAi oligonucleotide comprises an overhang of 2 nucleotides at the 3′-terminus of the sense and/or antisense strand, preferably of the antisense strand, more preferably a dTdT or a GG overhang at the 3′-terminus of the antisense strand or a 2-nucleotide overhang complementary to the KHK mRNA target sequence at the 3′-terminus of the antisense strand.

According to a specific embodiment, said sense strand and said antisense strand are separate strands, said sense strand is 21 nucleotides in length, said antisense strand is 23 nucleotides in length, said region of complementarity is 22 nucleotides in length, said duplex region is 21 nucleotides in length, and said dsRNAi oligonucleotide comprises a 2-nucleotide overhang complementary to the KHK mRNA target sequence at the 3′-terminus of the antisense strand.

According to another specific embodiment, said sense strand and said antisense strand are separate strands, said sense strand and said antisense strand are both 19 or 21 nucleotides in length, said region of complementarity is 19 nucleotides in length, said duplex region is 19 nucleotides in length, and said dsRNAi oligonucleotide optionally comprises a dTdT overhang at the 3′-terminus of the antisense strand.

According to another specific embodiment, said sense strand and said antisense strand are separate strands, said sense strand is 36 nucleotides in length, said antisense strand is 22 nucleotides in length, said region of complementarity is 19, 20, 21, or 22 nucleotides in length, said duplex region is 20 nucleotides in length, and said dsRNAi oligonucleotide comprises a GG overhang at the 3′-terminus of the antisense strand.

According to one embodiment, the dsRNAi oligonucleotide for reducing KHK expression comprises at least one modified nucleotide; preferably all of the nucleotides of the dsRNAi oligonucleotide for reducing KHK expression are modified.

According to another embodiment, the dsRNAi oligonucleotide for reducing KHK expression comprises at least one nucleotide with a modified sugar moiety, preferably selected from the group consisting of 2′-fluoro ribose and 2′-O-methyl ribose; e.g., it comprises 2′-fluoro modifications located at positions 1, 3, 5, 7, 9, 10, 11, 13, 15, 17, 19, and 21 from the 5′-end of the sense strand and/or at positions 2, 4, 6, 8, 10, 14, 16, 18, and 20 from the 5′-end of the antisense strand, while all the other nucleotide sugars are 2′-O-methyl modified; more preferably, it comprises not more than 11 2′-fluoro modifications, e.g., it comprises 2′-fluoro modifications located at positions 7, 9, 10, and 11 from the 5′-end of the sense strand and/or at positions 2, 14, and 16 from the 5′-end of the antisense strand, or it comprises 2′-fluoro modifications located at positions 8, 9, 10, and 11 from the 5′-end of the sense strand and/or at positions 2, 3, 4, 5, 7, 10, and 14 from the 5′-end of the antisense strand. According to another embodiment, the dsRNAi oligonucleotide for reducing KHK expression does not comprise any 2′-fluoro ribose modifications.

According to another embodiment, the dsRNAi oligonucleotide for reducing KHK expression comprises at least one glycol nucleic acid (GNA)-based nucleotide, preferably one GNA-based nucleotide located at position 7 from the 5′-end of the antisense strand.

According to another embodiment, the dsRNAi oligonucleotide for reducing KHK expression comprises at least one deoxyribonucleic acid (DNA)-nucleotide, preferably not more than 7 DNA nucleotides, e.g., located at positions 9 and 11 from the 5′-end of the sense strand and/or at positions 2, 5, 7, 12, and 14 from the 5′-end of the antisense strand.

According to another embodiment, the dsRNAi oligonucleotide for reducing KHK expression comprises at least one modified internucleotide linkage, preferably a phosphorothioate linkage, more preferably it comprises not more than 6 phosphorothioate linkages, e.g., 2 phosphorothioate linkages each at the 5′-ends of the sense and the antisense strand and at the 3′-end of the antisense strand, or 1 phosphorothioate linkage at the 5′-end of the sense strand and 3 phosphorothioate linkages at the 5′-end of the antisense strand and 2 phosphorothioate linkages at the 5′-end of the antisense strand.

According to another embodiment, the dsRNAi oligonucleotide for reducing KHK expression comprises a phosphate analog at the 5′-end of the antisense strand, preferably at the 4′-carbon of the sugar of the 5′-terminal nucleotide of the antisense strand, in particular 5′-methoxyphosphonate-4′-oxy, e.g., the 5′-terminal nucleotide of the antisense strand is 5′-methoxyphosphonate-4′-oxy-2′-O-methyluridine phosphorothioate ([MePhosphonate-4O-mUs] or [MePhosphonate-4O-mU]—S—):

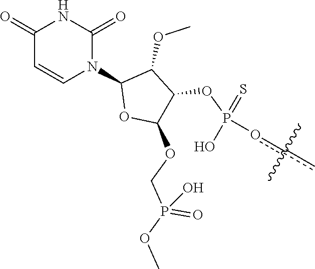

According to another embodiment, at least one nucleotide of the dsRNAi oligonucleotide is conjugated to an ASGPR targeting ligand, wherein each ASGPR targeting ligand comprises 1-3 N-acetylgalactosamine (GalNAc) moieties. Preferably, said at least one nucleotide of the dsRNAi oligonucleotide is comprised in the sense strand. More preferably, the sense strand comprises more than one GalNAc moiety conjugated via a monovalent, bivalent, trivalent, or tetravalent branched linker, e.g., 3 GalNAc moieties being conjugated either to one nucleotide of the sense strand via a trivalent branched linker, or to three nucleotides of the sense strand via monovalent linkers. In particular, the 3′-end of the sense strand may be conjugated via a trivalent branched linker to 3 GalNAc moieties (e.g., “L96”=N-[tris(GalNAc-alkyl)-amidodecanoyl)]-4-hydroxyprolinol, Hyp-(GalNAc-alkyl)3), e.g., as described in the prior art (e.g., WO 2021/178736), and exemplarily depicted below:

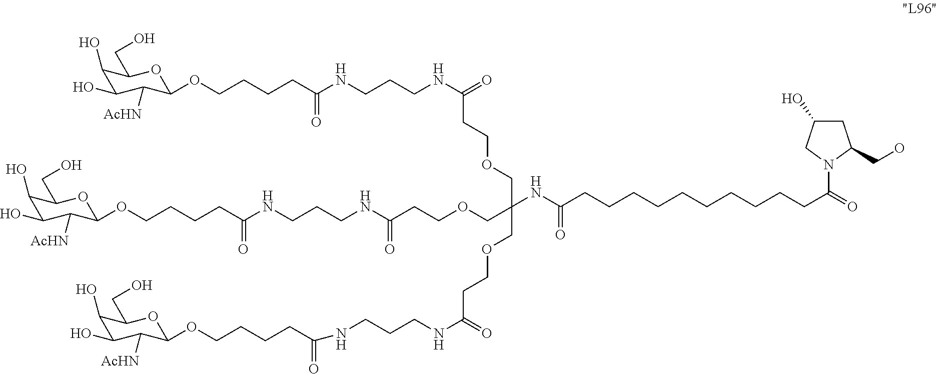 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O]

i.e., conjugated as

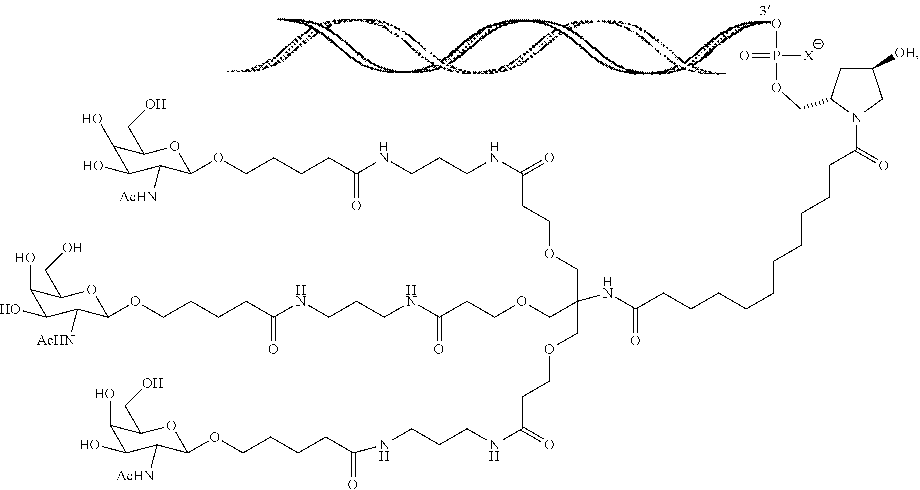

wherein X is O or S, preferably O.

Alternatively, the sense strand may comprise three 2′-aminodiethoxymethanol-Adenine-GalNAc nucleotides [ademA-GalNAc]:

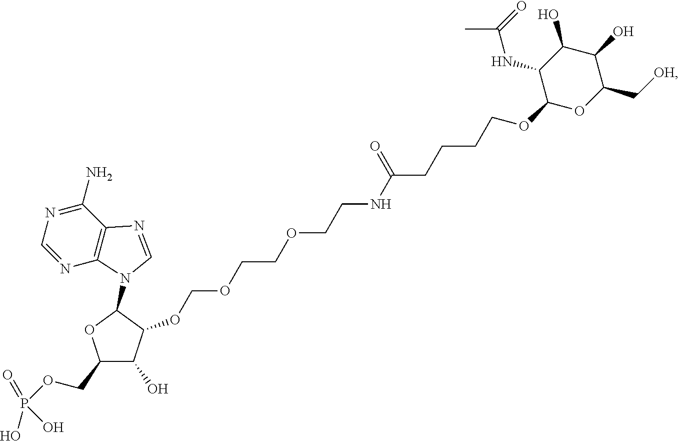 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

e.g., comprised within a tetraloop moiety of a stem-loop sequence located at the 3′-end of the sense strand.

According to one embodiment, the dsRNAi oligonucleotide for reducing KHK expression is selected from the group consisting of KHK-516, KHK-865, KHK-882, KHK-885, KHK-1078, and KHK-1334 as disclosed in U.S. Ser. No. 17/717,174 and as disclosed hereinbefore or hereinafter, which is incorporated by reference herein in its entirety.

According to another embodiment, the sense strand of the dsRNAi oligonucleotide for reducing KHK expression comprises a nucleotide sequence selected from the group consisting of
SEQ ID NO: 1 (KHK-516_ts);
SEQ ID NO: 2 (KHK-865_ts);
SEQ ID NO: 3 (KHK-882_ts);
SEQ ID NO: 4 (KHK-885_ts);
SEQ ID NO: 5 (KHK-1078_ts); and
SEQ ID NO: 6 (KHK-1334_ts).

According to another embodiment, the KHK mRNA target sequence comprises, preferably consists of, a nucleotide sequence selected from the group consisting of
SEQ ID NO: 1 (KHK-516_ta);
SEQ ID NO: 2 (KHK-865_ta);
SEQ ID NO: 3 (KHK-882_ta);
SEQ ID NO: 4 (KHK-885_ta);
SEQ ID NO: 5 (KHK-1078_ta); and
SEQ ID NO: 6 (KHK-1334_ta);
optionally wherein the region of complementarity is fully complementary to the KHK mRNA target sequence.

According to another embodiment, the sense and antisense strands of the dsRNAi oligonucleotide for reducing KHK expression comprise, preferably consist of, nucleotide sequences selected from the group consisting of
SEQ ID NOs: 7 and 13, respectively (KHK-516_s and KHK-516_a);
SEQ ID NOs: 8 and 14, respectively (KHK-865_s and KHK-865_a);
SEQ ID NOs: 9 and 15, respectively (KHK-882_s and KHK-882_a);
SEQ ID NOs: 10 and 16, respectively (KHK-885_s and KHK-885_a);
SEQ ID NOs: 11 and 17, respectively (KHK-1078_s and KHK-1078_a); and
SEQ ID NOs: 12 and 18, respectively (KHK-1334_s and KHK-1334_a);
optionally wherein all of the nucleotides of the dsRNAi oligonucleotide for reducing KHK expression are modified;
optionally wherein the dsRNAi oligonucleotide for reducing KHK expression comprises 2′-fluoro modifications located at positions 8, 9, 10, and 11 from the 5′-end of the sense strand and/or at positions 2, 3, 4, 5, 7, 10, and 14 from the 5′-end of the antisense strand;
optionally wherein at least one internucleotide linkage at the 5′-ends of each the sense and the antisense strand and/or at the 3′-end of the antisense strand is a phosphorothioate internucleotide linkage;
optionally wherein the dsRNAi oligonucleotide for reducing KHK expression comprises the phosphate analog 5′-methoxyphosphonate-4′-oxy at the 4′-carbon of the sugar of the 5′-terminal nucleotide of the antisense strand; and
optionally wherein the sense strand comprises three “ademA-GalNAc” nucleotides, preferably comprised within the tetraloop of the stem-loop sequence located at the 3′-end of the sense strand.

Within this invention, it is contemplated that the above-mentioned embodiments with regard to sequences and modifications may be combined with one another in light of the disclosures of the prior art, in particular to cover the following more specific embodiments.

According to another embodiment, the sense and antisense strands of the dsRNAi oligonucleotide for reducing KHK expression comprise, preferably consist of, nucleotide sequences including all of the modifications selected from the group consisting of
SEQ ID NOs: 19 and 25, respectively (KHK-516_sm and KHK-516_am);
SEQ ID NOs: 20 and 26, respectively (KHK-865_sm and KHK-865_am);
SEQ ID NOs: 21 and 27, respectively (KHK-882_sm and KHK-882_am);
SEQ ID NOs: 22 and 28, respectively (KHK-885_sm and KHK-885_am);
SEQ ID NOs: 23 and 29, respectively (KHK-1078_sm and KHK-1078_am); and
SEQ ID NOs: 24 and 30, respectively (KHK-1334_sm and KHK-1334_am).

According to another embodiment, the sense strand of the dsRNAi oligonucleotide for reducing KHK expression consists of the sequence and all of the modifications of mG-S-mA-mA-mG-mA-mG-mA-fA-fG-fC-fA-mG-mA-mU-mC-mC-mU-mG-mU-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 19), and the antisense strand of the dsRNAi oligonucleotide for reducing KHK expression consists of the sequence and all of the modifications of [MePhosphonate-4O-mU]-S-fA-S-fC-fA-fG-mG-fA-mU-mC-fU-mG-mC-mU-fU-mC-mU-mC-mU-mU-mC-S-mG-S-mG (5′->3′; SEQ ID NO: 25); or
the sense strand of the dsRNAi oligonucleotide for reducing KHK expression consists of the sequence and all of the modifications of mC-S-mA-mG-mA-mU-mG-mU-fG-fU-fC-fU-mG-mC-mU-mA-mC-mA-mG-mA-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 20), and the antisense strand of the dsRNAi oligonucleotide for reducing KHK expression consists of the sequence and all of the modifications of [MePhosphonate-4O-mU]-S-fU-S-fC-S-fU-fG-mU-fA-mG-mC-fA-mG-mA-mC-fA-mC-mA-mU-mC-mU-mG-S-mG-S-mG (5′->3′; SEQ ID NO: 26); or
the sense strand of the dsRNAi oligonucleotide for reducing KHK expression consists of the sequence and all of the modifications of -mG-S-mA-mC-mU-mU-mU-mG-fA-fG-fA-fA-mG-mG-mU-mU-mG-mA-mU-mC-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 21), and the antisense strand of the dsRNAi oligonucleotide for reducing KHK expression consists of the sequence and all of the modifications of [MePhosphonate-4O-mU]-S-fG-S-fA-S-fU-fC-mA-fA-mC-mC-fU-mU-mC-mU-fC-mA-mA-mA-mG-mU-mC-S-mG-S-mG (5′->3′; SEQ ID NO: 27); or
the sense strand of the dsRNAi oligonucleotide for reducing KHK expression consists of the sequence and all of the modifications of mU-S-mU-mU-mG-mA-mG-mA-fA-fG-fG-fU-mU-mG-mA-mU-mC-mU-mG-mA-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 22), and the antisense strand of the dsRNAi oligonucleotide for reducing KHK expression consists of the sequence and all of the modifications of [MePhosphonate-4O-mU]-S-fU-S-fC-S-fA-fG-mA-fU-mC-mA-fA-mC-mC-mU-fU-mC-mU-mC-mA-mA-mA-S-mG-S-mG (5′->3′; SEQ ID NO: 28); or
the sense strand of the dsRNAi oligonucleotide for reducing KHK expression consists of the sequence and all of the modifications of mU-S-mG-mU-mU-mU-mG-mU-fC-fA-fG-fC-mA-mA-mA-mG-mA-mU-mG-mU-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 23), and the antisense strand of the dsRNAi oligonucleotide for reducing KHK expression consists of the sequence and all of the modifications of [MePhosphonate-4O-mU]-S-fA-S-fC-fA-fU-mC-fU-mU-mU-fG-mC-mU-mG-fA-mC-mA-mA-mA-mC-mA-S-mG-S-mG (5′->3′; SEQ ID NO: 29); or

the sense strand of the dsRNAi oligonucleotide for reducing KHK expression consists of the sequence and all of the modifications of mG-S-mC-mA-mG-mG-mA-mA-fG-fC-fA-fC-mU-mG-mA-mG-mA-mU-mU-mC-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 24), and the antisense strand of the dsRNAi oligonucleotide for reducing KHK expression consists of the sequence and all of the modifications of [MePhosphonate-4O-mU]-S-fG-S-fA-S-fA-fU-mC-fU-mC-mA-fG-mU-mG-mC-fU-mU-mC-mC-mU-mG-mC-S-mG-S-mG (5′->3′; SEQ ID NO: 30);
wherein mC, mA, mG, mU=2′-OMe ribonucleosides; fA, fC, fG, fU=2′-F ribonucleosides; “-”=phosphodiester linkage, “—S—”=phosphorothioate linkage, and wherein [ademA-GalNAc] and [MePhosphonate-4O-mU]—S— are defined as hereinbefore.

According to another embodiment, the dsRNAi oligonucleotide for reducing KHK expression comprises a sense strand according to SEQ ID NO: 19 and an antisense strand according to SEQ ID NO: 25, wherein the dsRNAi oligonucleotide has the structure:

or
the dsRNAi oligonucleotide for reducing KHK expression comprises a sense strand according to SEQ ID NO: 20 and an antisense strand according to SEQ ID NO: 26, wherein the dsRNAi oligonucleotide has the structure:
   or
the dsRNAi oligonucleotide for reducing KHK expression comprises a sense strand according to SEQ ID NO: 21 and an antisense strand according to SEQ ID NO: 27, wherein the dsRNAi oligonucleotide
   or
the dsRNAi oligonucleotide for reducing KHK expression comprises a sense strand according to SEQ ID NO: 22 and an antisense strand according to SEQ ID NO: 28, wherein the dsRNAi oligonucleotide has the structure:
   or
the dsRNAi oligonucleotide for reducing KHK expression comprises a sense strand according to SEQ ID NO: 23 and an antisense strand according to SEQ ID NO: 29, wherein the dsRNAi oligonucleotide has the structure:
   or
the dsRNAi oligonucleotide for reducing KHK expression comprises a sense strand according to SEQ ID NO: 24 and an antisense strand according to SEQ ID NO: 30, wherein the dsRNAi oligonucleotide has the structure:

In a second aspect, the present invention relates to a pharmaceutical composition comprising one or more of said dsRNAi oligonucleotides, or one or more pharmaceutically acceptable salts thereof, and one or more pharmaceutically acceptable excipients for use in a method for the treatment of the advanced fibrotic and/or cirrhotic stages of NASH in a patient in need thereof.

It has been found that pharmaceutical compositions of dsRNAi oligonucleotides can be formulated that are suitable for the administration of therapeutically effective amounts of said dsRNAi oligonucleotides for the preventative and/or therapeutic treatment of the advanced fibrotic and/or cirrhotic stages of NASH. More specifically, dsRNAi oligonucleotide compositions can be formulated as solutions in aqueous media that are suitable for injection, in particular for subcutaneous injection. Such compositions provide therapeutically effective amounts of dsRNAi oligonucleotides when injected in physiologically and clinically acceptable application volumes. Appropriate formulation approaches, including the use of suitable excipients, conventional process steps and techniques are known to the one skilled in the art. Formulations of dsRNAi oligonucleotides are also described in WO 2015/123264, WO 2020/060986, WO 2021/178736, WO 2022/182574, and U.S. Ser. No. 17/717,174.

For instance, the dsRNAi oligonucleotide may be precipitated, redissolved in water, and lyophilized The dried dsRNAi oligonucleotides may then be dissolved in an aqueous medium, e.g., in isotonic saline (0.90% w/v of sodium chloride), to form the pharmaceutical composition for injection. Inorganic hydroxides (like alkali hydroxides and alkaline earth hydroxides) and/or inorganic acids, in particular sodium hydroxide and/or concentrated phosphoric acid, may be used to adjust the pH of the solution to physiologically acceptable values. Also, the osmolality of the final solution for injection should be in a physiologically acceptable range.

The concentration of the dsRNAi oligonucleotide in the composition should be chosen to allow for sufficient reduction of KHK expression in the liver when a physiologically and clinically acceptable amount of the composition, i.e., a therapeutically effective dose of the dsRNAi oligonucleotide, is administered.

According to one embodiment, the pharmaceutical composition is an aqueous solution, preferably for subcutaneous injection, of one or more dsRNAi oligonucleotides for reducing KHK expression as described herein.

According to another embodiment, the aqueous solution comprises 1 or 2 dsRNAi oligonucleotides for reducing KHK expression as described herein, or pharmaceutically acceptable salts thereof, preferably only one dsRNAi oligonucleotide for reducing KHK expression, or a pharmaceutically acceptable salt thereof.

According to another embodiment, the pharmaceutical composition is a solution of one or more, preferably one, dsRNAi oligonucleotides for reducing KHK expression as described herein in isotonic saline.

According to another embodiment, the aqueous solution comprises, preferably consists of, one or more dsRNAi oligonucleotides for reducing KHK expression, or pharmaceutically acceptable salts thereof, as active pharmaceutical ingredient(s) as well as water for injection, alkali hydroxides, e.g., sodium hydroxide, and inorganic acids, e.g., phosphoric acid, as excipients.

According to another embodiment, the application volume of the aqueous solution for subcutaneous injection is not more than about 3 mL, preferably not more than about 2 mL, more preferably not more than about 1 mL.

According to another embodiment, the concentration of each dsRNAi oligonucleotide comprised by the aqueous solution is in the range from about 1 mg/mL to about 1 g/mL, preferably in the range from about 10 mg/mL to about 500 mg/mL, more preferably in the range from about 50 mg/mL to about 200 mg/mL, e.g., about 190 mg/mL.

According to another embodiment, the pH value of the aqueous solution is physiologically acceptable, e.g., approximately 7.0.

According to another embodiment, the osmolality of the aqueous solution is in the physiologically acceptable range, e.g., in the range from approximately 210 mOsm/kg to approximately 390 mOsm/kg, more specifically from approximately 270 mOsm/kg to approximately 330 mOsm/kg, e.g., from approximately 275 mOsm/kg to approximately 295 mOsm/kg.

According to another embodiment, the pharmaceutical composition, is administered in combination with a second composition suitable for the treatment of NASH.

Likewise, the present invention relates to a method for the treatment of the advanced fibrotic and/or cirrhotic stages of NASH in a patient in need thereof, the method being characterized in that one or more of said dsRNAi oligonucleotides, or one or more pharmaceutically acceptable salts thereof, and/or one or more of said pharmaceutical compositions is administered to the patient.

Furthermore, the present invention relates to a method for the treatment of the advanced fibrotic and/or cirrhotic stages of NASH with one or more of the above-mentioned pharmaceutical compositions.

Said method is characterized by the features and embodiments described above for the first and second aspects of the present invention.

Likewise, the present invention relates to the use of one or more of said dsRNAi oligonucleotides, or one or more pharmaceutically acceptable salts thereof, in the manufacture of a medicament for the treatment of the advanced fibrotic and/or cirrhotic stages of NASH in a patient in need thereof.

Said medicament and said method are characterized by the features and embodiments described above for the first and second aspects of the present invention.

Further aspects of the present invention will become apparent to the person skilled in the art directly from the foregoing and following description, as well as from the Examples described herein.

In Example 2 was used a mouse active GalNAc-conjugated KHK oligonucleotide named Compound A. Compound A is dsRNAi oligonucleotide comprising a sense strand according to SEQ ID NO: 34 and an antisense strand according to Example 4, “Compound A_am”, wherein the dsRNAi oligonucleotide has the structure:

### EXAMPLES

The following Examples are offered by way of illustration and are not intended to limit the scope of the disclosure in any manner. In addition, modifications may be made to adapt to a situation, material, composition of matter, process, process step or steps, to the objective, spirit, and scope of the disclosure. All such modifications are intended to be within the scope of the disclosure. Standard techniques well known in the art or the techniques specifically described below were utilized.

### Example 1: Preparation of dsRNAi Oligonucleotides

### Oligonucleotide Synthesis and Purification

The dsRNAi oligonucleotides described hereinbefore or hereinafter may be chemically synthesized using methods described herein as well as in WO 2015/123264, WO 2020/060986, WO 2021/178736, WO 2022/182574, U.S. Ser. No. 17/717,174, WO 2018/045317, and WO 2016/100401, which are all incorporated by reference herein in their entireties.

Generally, dsRNAi oligonucleotides are synthesized using solid phase oligonucleotide synthesis methods as described for 19-23 mer siRNAs (see, e.g., Scaringe et al. (1990) Nucleic Acids Res. 18:5433-5441 and Usman et al. (1987) J. Am. Chem. Soc. 109:7845-7845; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis (see, e.g., Hughes and Ellington (2017) COLD SPRING HARB PERSPECT BIOL. 9(1):a023812; Beaucage S.L., Caruthers M.H., Studies on Nucleotide Chemistry V: Deoxynucleoside Phosphoramidites—A New Class of Key Intermediates for Deoxypolynucleotide Synthesis, TETRAHEDRON LETT. 1981; 22:1859-62. doi: 10.1016/50040-4039(01)90461-7).

Individual RNA strands are synthesized and HPLC purified according to standard methods (Integrated DNA Technologies; Coralville, IA). For example, RNA oligonucleotides are synthesized using solid phase phosphoramidite chemistry, deprotected and desalted on NAP-5 columns (Amersham Pharmacia Biotech; Piscataway, NJ) using standard techniques (Damha & Olgivie (1993) Methods Mol. Biol. 20:81-114; Wincott et al. (1995) Nucleic Acids Res. 23:2677-84). The oligomers are purified using ion-exchange high performance liquid chromatography (IE-HPLC) on an Amersham Source 15Q column (1.0 cm×25 cm; Amersham Pharmacia Biotech) using a 15 mM step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples are monitored at 260 nm and peaks corresponding to the full-length oligonucleotide species are collected, pooled, desalted on NAP-5 columns, and lyophilized

The purity of each oligomer is determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmol of oligonucleotide is injected into a capillary, run in an electric field of 444 V/cm, and detected by UV absorbance at 260 nm. Denaturing Tris-Borate-7 M-urea running buffer is purchased from Beckman-Coulter. Oligoribonucleotides are obtained that are at least 90% pure as assessed by CE for use in experiments described below. Compound identity is verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all oligomers are obtained, often within 0.2% of expected molecular mass.

### Preparation of Duplexes

Single strand RNA oligomers are resuspended (e.g., at 100 μM concentration) in duplex buffer consisting of 100 mM potassium acetate, 30 mM HEPES, pH 7.5. Complementary sense and antisense strands are mixed in equal molar amounts to yield a final solution of, for example, 50 μM duplex. Samples are heated to 100° C. for 5′ in RNA buffer (IDT) and are allowed to cool to room temperature before use. The dsRNA oligonucleotides are stored at −20° C. Single strand RNA oligomers are stored lyophilized or in nuclease-free water at −80° C.

### Example 2: Anti-fibrotic Efficacy of an RNAi Oligonucleotide Targeting KHK in a Mouse Model

A mouse active GalNAc-conjugated KHK oligonucleotide (“Compound A”, SEQ ID NO 34 and Compound A_am), which had been found to effectively knock-down KHK in the mouse liver, was investigated on metabolic parameters and liver histopathology in a biopsy-confirmed and diet-induced obese (DIO) mouse model of NASH.

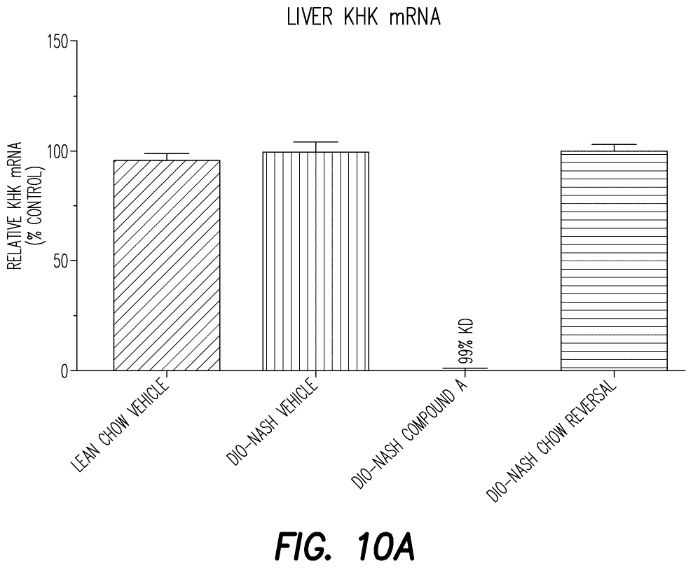

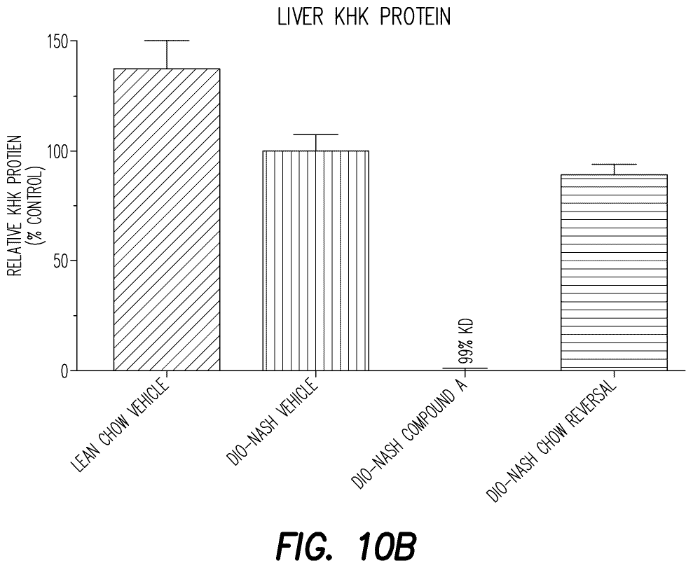

Male C57BL/6 mice (n=50) were fed the Gubra AMLN NASH (GAN) diet (Hansen et al., BMC Gastroenterology 2020, 20, 210) or Altromin Chow (n=6) for 32 weeks before treatment start. The GAN diet mouse model develops fibrosis which represents a preclinical model for human NASH. This model has been used to investigate the effect of KHK knock-down using Compound A in an interventive setting Prior to treatment, all animals underwent liver biopsy for histological confirmation (steatosis score ≥2 and fibrosis stage ≥1). Mice were stratified into treatment groups based on quantitative liver fibrosis staining (Picro-Sirius Red, PSR)). DIO-NASH (n=14/group) mice received vehicle or Compound A (6 mg/kg s.c. on days 0 and 2; 3 mg/kg s.c. once weekly in weeks 2-11) for 12 weeks. Chow-fed mice (n=6) and DIO-NASH Chow Reversal (n=14) animals served as controls and received vehicle s.c. using the same dose intervals. Treatment with Compound A resulted in a 99% KHK protein knock-down (FIG. 10A-10B). Endpoints included metabolic and histopathological scores and histomorphometry.

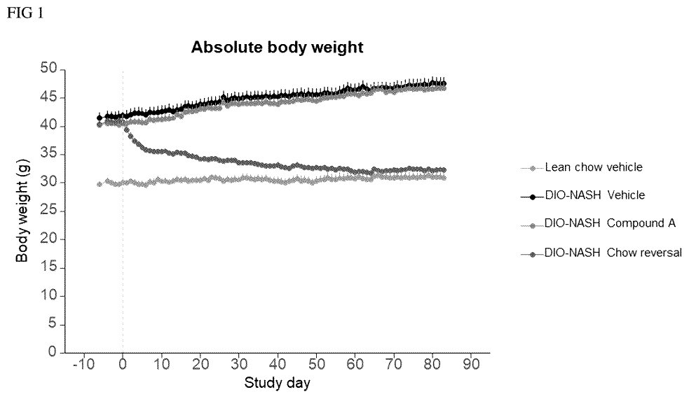

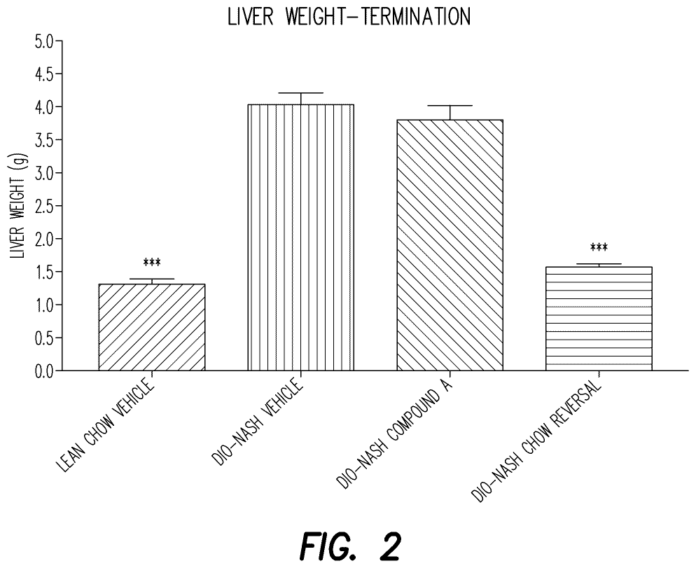

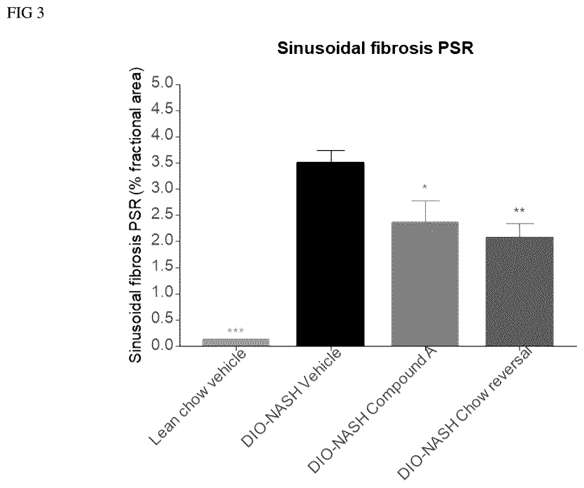

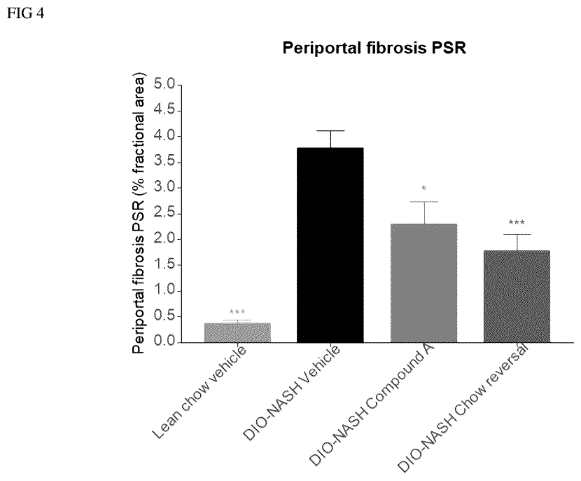

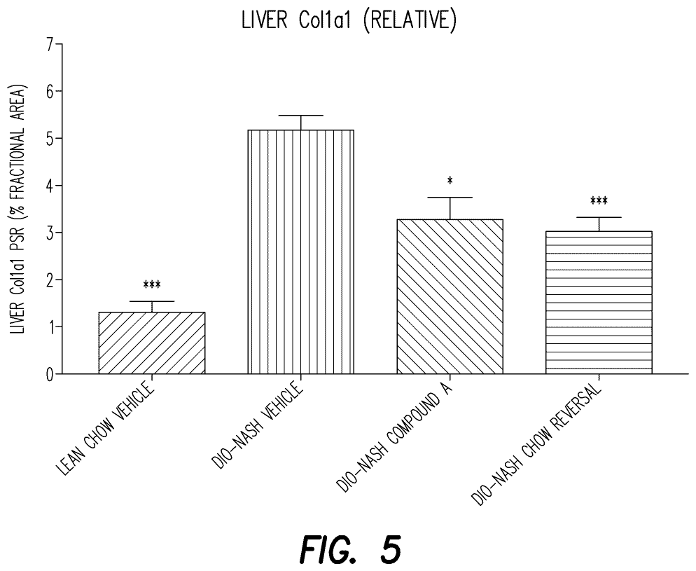

Compared to vehicle dosing, Compound A did not influence body or liver weight in DIO-NASH mice (FIG. 1-2). Compound A showed no significant effect on histopathological scores. Histomorphometric analyses indicated that Compound A reduced % area of sinusoidal and periportal fibrosis as well as Col1a1 (FIG. 3-5). Compound A increased quantitative markers of steatosis (% area of liver lipid) but did not change quantitative markers of inflammation (number of inflammatory cells/foci, galectin-3) and hepatic stellate cell activation (α-SMA). DIO-NASH Chow Reversal significantly reduced metabolic (body and liver weight) and histological parameters including NAFLD Activity Score, liver steatosis (steatosis score, % area of liver lipid), liver inflammation (inflammation score, % area of inflammatory cells/foci, galectin-3), and fibrosis (% area of PSR, % area of sinusoidal/periportal fibrosis, Coll al and α-SMA).

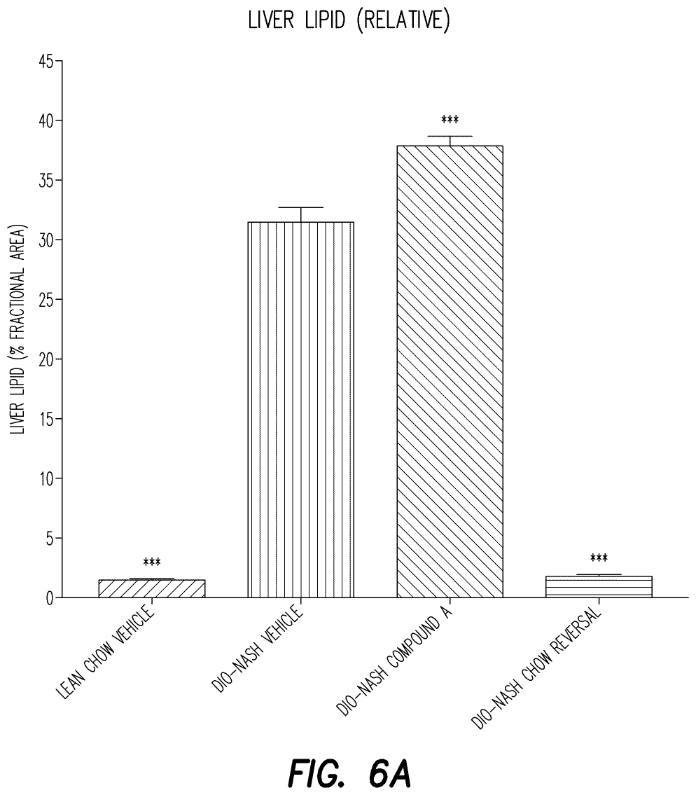

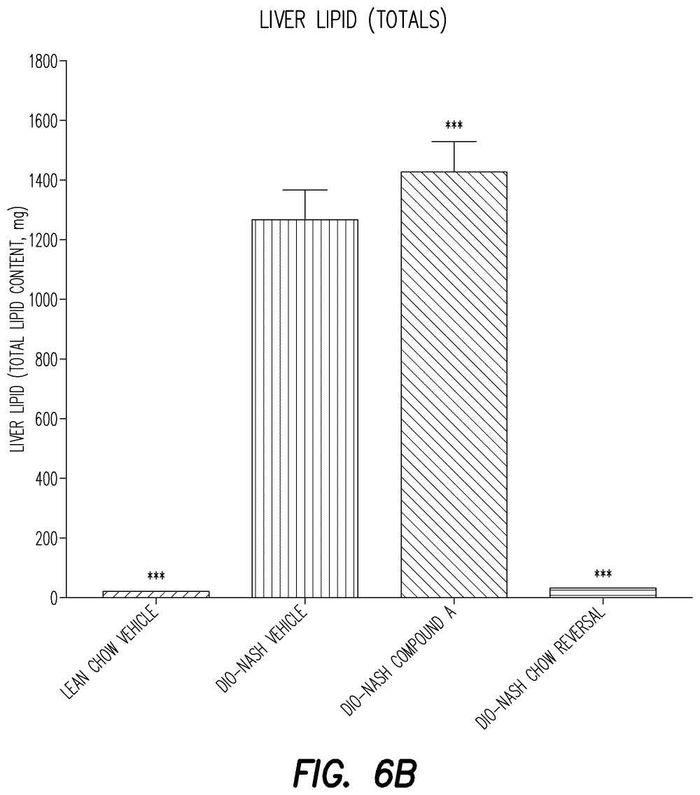

Compound A increased liver lipid accumulation (FIG. 6), without affecting steatosis scores, and reduced histomorphometric variables of liver fibrosis in DIO-NASH mice. Chow Reversal improved NASH histopathology by reducing both qualitative and quantitative parameters of steatosis, inflammation, and fibrosis.

Altogether, KHK knock down in mice using a KHK-targeting GalNAc-siRNA resulted in a strong reduction of histologically proven fibrosis.

### Example 3: RNAi Oligonucleotide Inhibition of KHK Expression (Single-Dose Studies in Non-human Primates)

### Single-Dose Non-Human Primate (NHP) Studies

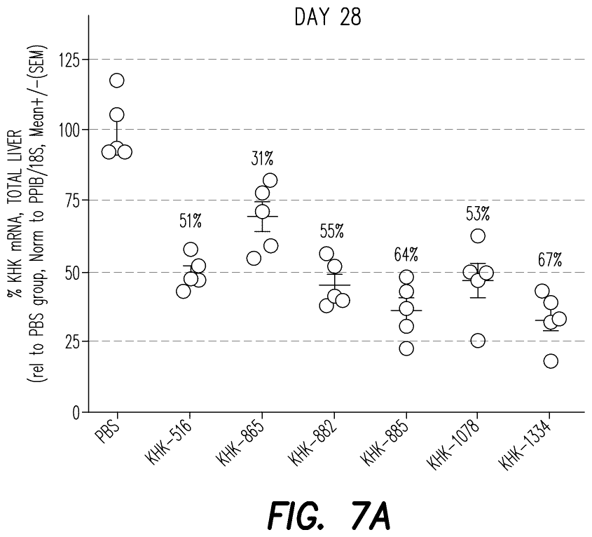

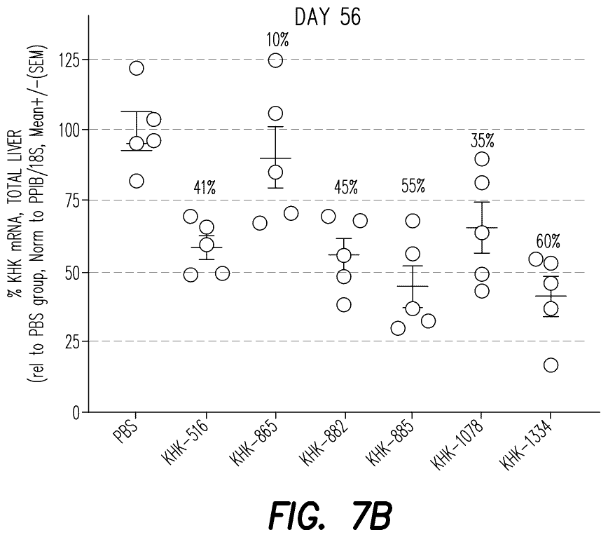

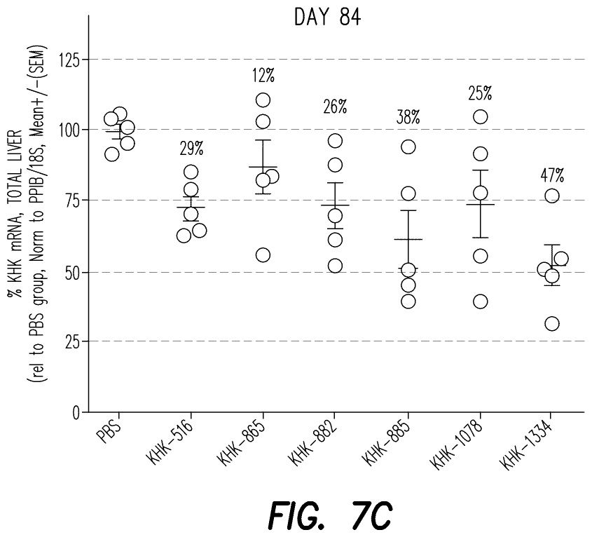

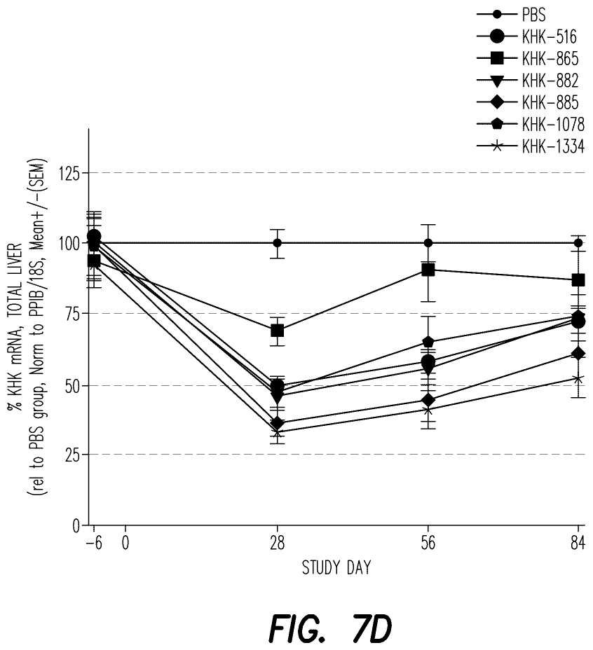

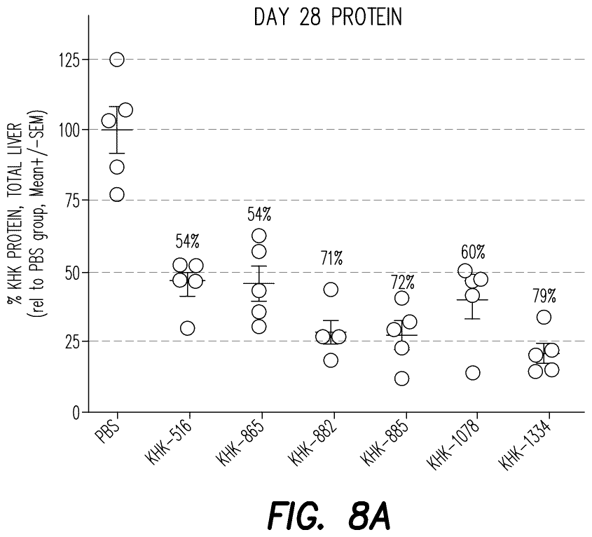

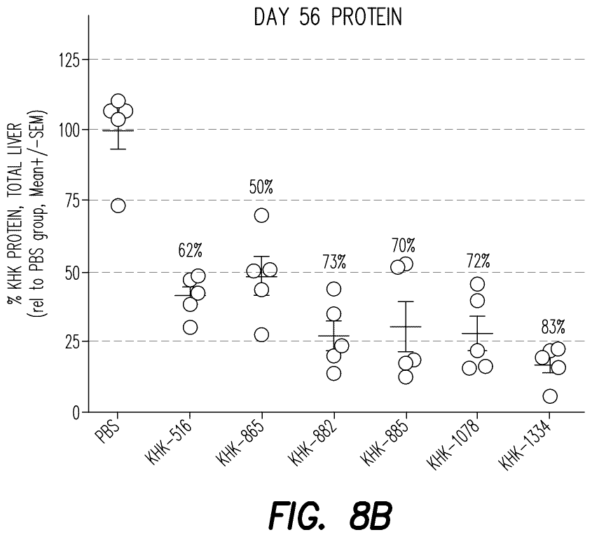

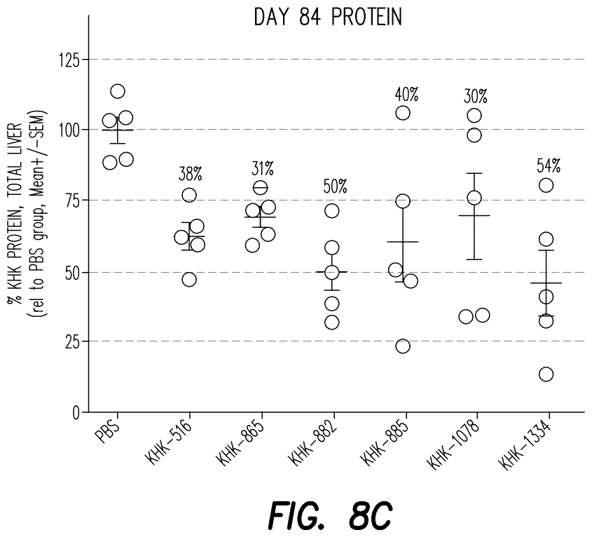

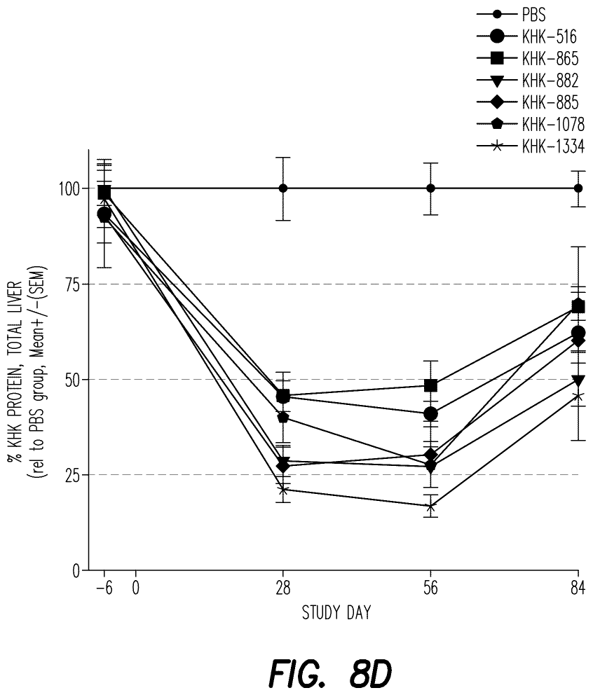

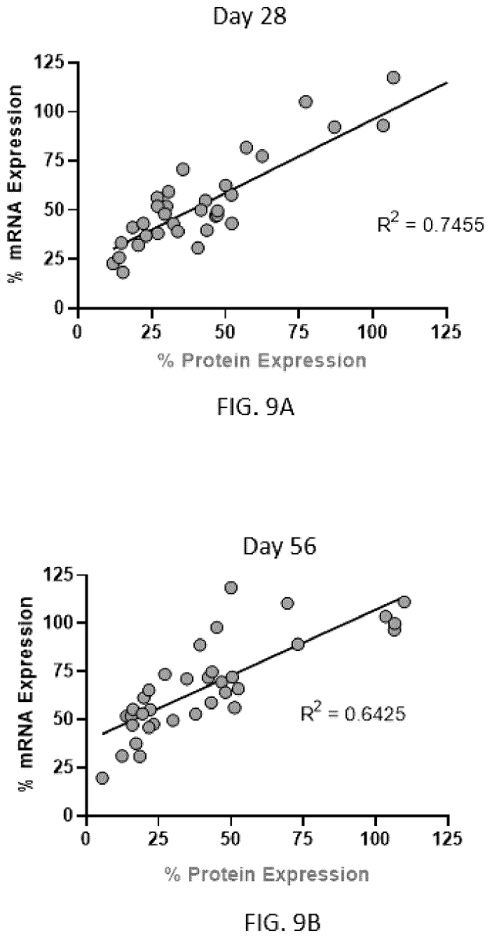

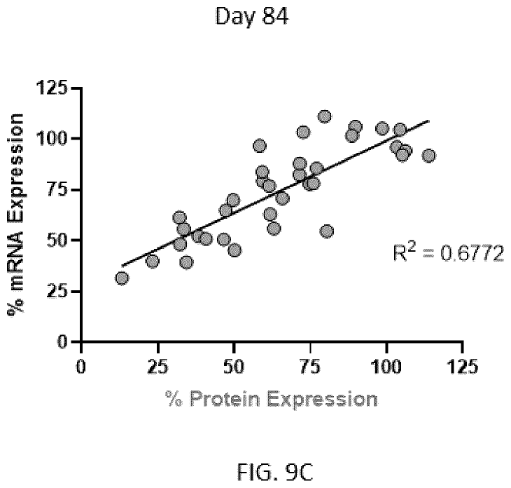

The GalNAc-conjugated KHK oligonucleotides listed in Table 1 were evaluated in non-naïve cynomolgus monkeys (Macaca fascicularis). In this study, the monkeys were grouped so that their mean body weights (about 5.4 kg) were comparable between the control and experimental groups. Each cohort contained at least two female and at least two male subjects. The GalNAc-conjugated KHK oligonucleotides were administered subcutaneously at a dose of 6 mg/kg on Study Day 0. Blood samples were collected one week prior to dosing (Day −7), on the dosing date (Day 0) and days 28, 56 and 84 after dosing. Ultrasound-guided core needle liver biopsies were collected on Study Days −7, 28, 56 and 84. At each time point, total RNA derived from the liver biopsy samples was subjected to qRT-PCR analysis to measure KHK mRNA in oligonucleotide-treated monkeys relative to those treated with a comparable volume of PBS. To normalize the data, the measurements were made relative to the geometric mean of two reference genes, PPIB and 18S rRNA. The following TaqMan qPCR probes purchased from Life Technologies, Inc, were used to evaluate gene expressions: Forward—TGCCTTCATGGGCTCAATG (SEQ ID NO: 31); Reverse—TCGGCCACCAGGAAGTCA (SEQ ID NO: 32); Fam probe-CCCTGGCCATGTTG (SEQ ID NO:33). As shown in FIG. 7A (Day 28), treating NHPs with the GalNAc-conjugated KHK oligonucleotides listed in Table 1 inhibited KHK expression in the liver, as determined by a reduced amount of KHK mRNA in liver samples from oligonucleotide-treated NHPs relative to NHPs treated with PBS. The mean percent reduction of KHK mRNA in the liver samples of treated NHPs is indicated above the set of data points for each treatment group. Days 56 and 84 were also measured (FIG. 7B and 7C) and a plot of the mean values over each time point is shown in FIG. 7D. For all time points evaluated, almost all the tested GalNAc-conjugated KHK oligonucleotides significantly inhibited KHK mRNA expression. In the same samples, KHK protein levels were detected using rabbit anti-Ketohexokinase (Abcam, AB197593) and anti-rabbit Detection Module for Sally Sue (Protein Simple, cat #DM-001). As shown in FIGS. 8A-8C, at the 28-day timepoint, GalNAc-KHK constructs inhibit KHK protein expression, as normalized to the vinculin control and slowly increases by Day 86. These results demonstrate that treating NHPs with the GalNAc-conjugated KHK oligonucleotides reduces the amount of KHK mRNA in the liver and concomitantly reduces the amount of KHK protein in the liver. However, this correlation is reduced over time after the initial dose (FIGS. 9A-9C).

Taken together, these results show that GalNAc-conjugated KHK oligonucleotides designed to target human total KHK mRNA inhibit total KHK expression in vivo (as determined by the reduction of the amount of KHK mRNA and protein).

TABLE 1
GalNAc-KHK Constructs for Single-dose NHP Study

| | Sense strand | Anti-sense strand |
|---|---|---|
| Name | SEQ ID NO | SEQ ID NO |
| KHK-516 | 19 | 25 |
| KHK-865 | 20 | 26 |
| KHK-882 | 21 | 27 |
| KHK-885 | 22 | 28 |
| KHK-1078 | 23 | 29 |
| KHK-1334 | 24 | 30 |

### Example 4: Pharmaceutical Formulations

An exemplary, non-limiting pharmaceutical formulation suitable for subcutaneous injection is described by the following composition:

| Ingredient | Amount | |
|---|---|---|
| dsRNAi | 190 | mg |
| oligonucleotide | | |
| (as described herein) | | |
| water for injection | 1 | mL |
| sodium chloride | 9.0 | mg |

SEQUENCE LISTING

| SEQ ID NO | Name | Sequence (5′ -> 3′) |
|---|---|---|
| 1 | KHK-516_ts, | GAAGAGAAGCAGAUCCUGU |
| | KHK-516_ta | |
| 2 | KHK-865_ts, | CAGAUGUGUCUGCUACAGA |
| | KHK-865_ta | |
| 3 | KHK-882_ts, | GACUUUGAGAAGGUUGAUC |
| | KHK-882_ta | |
| 4 | KHK-885_ts, | UUUGAGAAGGUUGAUCUGA |
| | KHK-885_ta | |
| 5 | KHK-1078_ts, | UGUUUGUCAGCAAAGAUGU |
| | KHK-1078_ta | |
| 6 | KHK-1334_ts, | GCAGGAAGCACUGAGAUUC |
| | KHK-1334_ta | |
| 7 | KHK-516_s | GAAGAGAAGCAGAUCCUGUAGCAGCCGAAAGGCUGC |
| 8 | KHK-865_s | CAGAUGUGUCUGCUACAGAAGCAGCCGAAAGGCUGC |
| 9 | KHK-882_s | GACUUUGAGAAGGUUGAUCAGCAGCCGAAAGGCUGC |
| 10 | KHK-885_s | UUUGAGAAGGUUGAUCUGAAGCAGCCGAAAGGCUGC |
| 11 | KHK-1078_s | UGUUUGUCAGCAAAGAUGUAGCAGCCGAAAGGCUGC |
| 12 | KHK-1334_s | GCAGGAAGCACUGAGAUUCAGCAGCCGAAAGGCUGC |
| 13 | KHK-516_a | UACAGGAUCUGCUUCUCUUCGG |
| 14 | KHK-865_a | UUCUGUAGCAGACACAUCUGGG |
| 15 | KHK-882_a | UGAUCAACCUUCUCAAAGUCGG |
| 16 | KHK-885_a | UUCAGAUCAACCUUCUCAAAGG |
| 17 | KHK-1078_a | UACAUCUUUGCUGACAAACAGG |
| 18 | KHK-1334_a | UGAAUCUCAGUGCUUCCUGCGG |
| 19 | KHK-516_sm | [mGs][mA][mA][mG][mA][mG][mA][fA][fG][fC][fA][mG] |
| | | [mA][mU][mC][mC][mU][mG][mU][mA][mG][mC][mA][mG] |
| | | [mC][mC][mG][ademA-GalNAc][ademA-GalNAc][ademA- |
| | | GalNAc][mG][mG][mC][mU][mG][mC] |
| 20 | KHK-865_sm | [mCs][mA][mG][mA][mU][mG][mU][fG][fU][fC][fU][mG] |
| | | [mC][mU][mA][mC][mA][mG][mA][mA][mG][mC][mA][mG] |
| | | [mC][mC][mG][ademA-GalNAc][ademA-GalNAc] [ademA- |
| | | GalNAc][mG][mG][mC][mU][mG][mC] |
| 21 | KHK-882_sm | [mGs][mA][mC][mU][mU][mU][mG][fA][fG][fA][fA][mG] |
| | | [mG][mU][mU][mG][mA][mU][mC][mA][mG][mC][mA][mG] |
| | | [mC][mC][mG][ademA-GalNAc][ademA-GalNAc][ademA- |
| | | GalNAc][mG][mG][mC][mU][mG][mC] |
| 22 | KHK-885_sm | [mUs][mU][mU][mG][mA][mG][mA][fA][fG][fG][fU][mU] |
| | | [mG][mA][mU][mC][mU][mG][mA][mA][mG][mC][mA][mG] |
| | | [mC][mC][mG][ademA-GalNAc][ademA-GalNAc][ademA- |
| | | GalNAc][mG][mG][mC][mU][mG][mC] |
| 23 | KHK-1078_sm | [mUs][mG][mU][mU][mU][mG][mU][fC][fA][fG][fC][mA] |
| | | [mA][mA][mG][mA][mU][mG][mU][mA][mG][mC][mA][mG] |
| | | [mC][mC][mG][ademA-GalNAc][ademA-GalNAc][ademA- |
| | | GalNAc][mG][mG][mC][mU][mG][mC] |
| 24 | KHK-1334_sm | [mGs][mC][mA][mG][mG][mA][mA][fG][fC][fA][fC][mU] |
| | | [mG][mA][mG][mA][mU][mU][mC][mA][mG][mC][mA][mG] |
| | | [mC][mC][mG][ademA-GalNAc][ademA-GalNAc][ademA- |
| | | GalNAc][mG][mG][mC][mU][mG][mC] |
| 25 | KHK-516_am | [MePhosphonate-40-mUs][fAs][fC][fA][fG][mG][fA] |
| | | [mU][mC][fU][mG][mC][mU][fU][mC][mU][mC][mU][mU] |
| | | [mCs][mGs][mG] |
| 26 | KHK-865_am | [MePhosphonate-40-mUs][fUs][fCs][fU][fG][mU][fA] |
| | | [mG][mC][fA][mG][mA][mC][fA][mC][mA][mU][mC][mU] |
| | | [mGs][mGs][mG] |
| 27 | KHK-882_am | [MePhosphonate-40-mUs][fGs][fAs][fU][fC][mA][fA] |
| | | [mC][mC][fU][mU][mC][mU][fC][mA][mA][mA][mG][mU] |
| | | [mCs][mGs][mG] |
| 28 | KHK-885_am | [MePhosphonate-40-mUs][fUs][fCs][fA][fG][mA][fU] |
| | | [mC][mA][fA][mC][mC][mU][fU][mC][mU][mC][mA][mA] |
| | | [mAs][mGs][mG] |
| 29 | KHK-1078_am | [MePhosphonate-40-mUs][fAs][fC][fA][fU][mC][fU] |
| | | [mU][mU][fG][mC][mU][mG][fA][mC][mA][mA][mA][mC] |
| | | [mAs][mGs][mG] |
| 30 | KHK-1334_am | [MePhosphonate-40-mUs][fGs][fAs][fA][fU][mC][fU] |
| | | [mC][mA][fG][mU][mG][mC][fU][mU][mC][mC][mU][mG] |
| | | [mCs][mGs][mG] |
| 31 | Forward probe | TGCCTTCATGGGCTCAATG |
| 32 | Reverse probe | TCGGCCACCAGGAAGTCA |
| 33 | Fam probe | CCCTGGCCATGTTG |
| 34 | Compound A_sm | [mAs][mG][fU][mU][mG][mU][mU][fU][fA][fG][mC][fU] |
| | | [fA][mU][mG][mG][fU][mG][mA][mA][mG][mC][mA][mG] |
| | | [mC][mC][mG][ademA-GalNAc][ademA-GalNAc][ademA- |
| | | GalNAc][mG][mG][mC][mU][mG][mC] |
| n/a | Compound A_am | [MePhosphonate-40-mUs][fUs][mCs][mA][fC][mC][fA] |
| | | [fU][mA][fG][mC][fU][mA][fA][mA][fC][mA][mA][fC] |
| | | [mUs][mGs][mG] |

Modification Key

| | |
|---|---|
| Symbol | Modification/linkage |

Key 1

| | |
|---|---|
| X | nucleoside-3′-phosphate |
| for X = A | nucleoside = adenosine |
| for X = C | nucleoside = cytidine |
| for X = G | nucleoside = guanosine |
| for X = T | nucleoside = thymidine = 5′-methyluridine |
| for X = U | nucleoside = uridine |

Key 2

| | |
|---|---|
| [fX] | 2′-fluoro modified nucleotide with phosphodiester linkages to neighboring |
| | nucleotides |
| [mX] | 2′-O-methyl modified nucleotide with phosphodiester linkages to |
| | neighboring nucleotides |
| [fXs] | 2′-fluoro modified nucleotide with a phosphorothioate linkage to the |
| | neighboring nucleotide |
| [mXs] | 2′-O-methyl modified nucleotide with a phosphorothioate linkage to the |
| | neighboring nucleotide |
| [MePhosphonate- | 5′-methoxyphosphonate-4′-oxy modified [mUs] |
| 4O-mUs] | 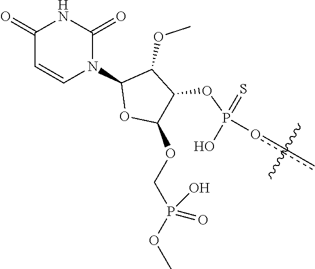 |
| [ademA-GalNAc] | 2′-aminodiethoxymethanol-Adenine-GalNAc with phosphodiester linkages |
| | to neighboring nucleotides |
| | 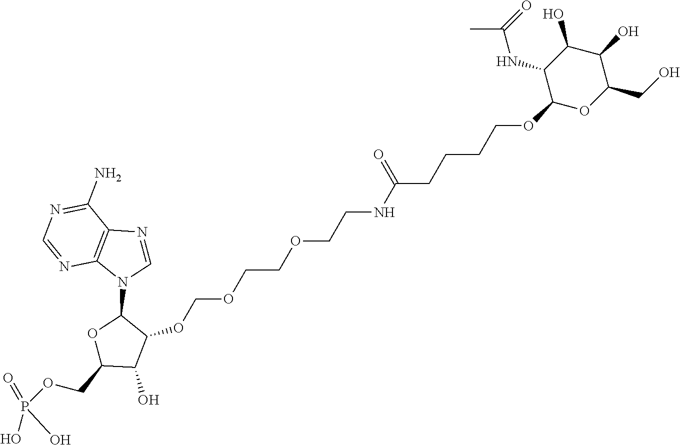 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O] |

1. A method for the treatment of non-alcoholic steatohepatitis (NASH) in a patient in need thereof, comprising administration of a double stranded RNAi (dsRNAi) oligonucleotide, or a pharmaceutically acceptable salt thereof, to the patient,
wherein the dsRNAi oligonucleotide comprises an antisense strand and a sense strand, wherein the antisense strand and the sense strand form a duplex region, and wherein the antisense strand comprises a region of complementarity to a KHK mRNA target sequence;
wherein the KHK mRNA target sequence comprises a nucleotide sequence selected from the group consisting of
a) SEQ ID NO: 4 (KHK-885_ta);
b) SEQ ID NO: 6 (KHK-1334_ta);
c) SEQ ID NO: 1 (KHK-516_ta);
d) SEQ ID NO: 2 (KHK-865_ta);
e) SEQ ID NO: 3 (KHK-882_ta); and
f) SEQ ID NO: 5 (KHK-1078_ta);
and
wherein the region of complementarity is fully complementary to the KHK mRNA target sequence, wherein the dsRNAi is capable of reducing ketohexokinase (KHK) expression and wherein NASH is in the advanced fibrotic and/or cirrhotic stage.

2. The method according to claim 1, wherein the patient's NASH is in the advanced fibrotic stage.

3. The method according to claim 1, wherein the patient's NASH is in the cirrhotic stage.

4. The method of claim 1, wherein
a) the sense strand of the dsRNAi oligonucleotide consists of the sequence and all of the modifications of mU-S-mU-mU-mG-mA-mG-mA-fA-fG-fG-fU-mU-mG-mA-mU-mC-mU-mG-mA-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 22), and the antisense strand of the dsRNAi oligonucleotide for reducing KHK expression consists of the sequence and all of the modifications of [MePhosphonate-4O-mU]-S-fU-S-fC-S-fA-fG-mA-fU-mC-mA-fA-mC-mC-mU-fU-mC-mU-mC-mA-mA-mA-S-mG-S-mG (5′->3′; SEQ ID NO: 28);
or
b) the sense strand of the dsRNAi oligonucleotide consists of the sequence and all of the modifications of mG-S-mC-mA-mG-mG-mA-mA-fG-fC-fA-fC-mU-mG-mA-mG-mA-mU-mU-mC-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 24), and the antisense strand of the dsRNAi oligonucleotide for reducing KHK expression consists of the sequence and all of the modifications of [MePhosphonate-4O-mU]-S-fG-S-fA-S-fA-fU-mC-fU-mC-mA-fG-mU-mG-mC-fU-mU-mC-mC-mU-mG-mC-S-mG-S-mG (5′ ->3′ ; SEQ ID NO: 30);
or
c) the sense strand of the dsRNAi oligonucleotide consists of the sequence and all of the modifications of mG-S-mA-mA-mG-mA-mG-mA-fA-fG-fC-fA-mG-mA-mU-mC-mC-mU-mG-mU-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 19), and the antisense strand of the dsRNAi oligonucleotide for reducing KHK expression consists of the sequence and all of the modifications of [MePhosphonate-4O-mU]-S-fA-S-fC-fA-fG-mG-fA-mU-mC-fU-mG-mC-mU-fU-mC-mU-mC-mU-mU-mC-S-mG-S-mG (5′->3′; SEQ ID NO: 25);
or
d) the sense strand of the dsRNAi oligonucleotide consists of the sequence and all of the modifications of mC-S-mA-mG-mA-mU-mG-mU-mG-fU-fC-fU-mG-mC-mU-mA-mC-mA-mG-mA-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 20), and the antisense strand of the dsRNAi oligonucleotide for reducing KHK expression consists of the sequence and all of the modifications of [MePhosphonate-4O-mU]-S-fU-S-fC-S-fU-fG-mU-fA-mG-mC-fA-mG-mA-mC-fA-mC-mA-mU-mC-mU-mG-S-mG-S-mG (5′->3′; SEQ ID NO: 26); or
e) the sense strand of the dsRNAi oligonucleotide consists of the sequence and all of the modifications of -mG-S-mA-mC-mU-mU-mU-mG-fA-fG-fA-fA-mG-mG-mU-mU-mG-mA-mU-mC-mA-mG-mC-mA-mG-mC-mC-mG-[ademA-GalNAc]-[ademA-GalNAc]-[ademA-GalNAc]-mG-mG-mC-mU-mG-mC (5′ ->3′ ; SEQ ID NO: 21), and the antisense strand of the dsRNAi oligonucleotide for reducing KHK expression consists of the sequence and all of the modifications of [MePhosphonate-4O-mU]-S-fG-S-fA-S-fU-fC-mA-fA-mC-mC-fU-mU-mC-mU-fC-mA-mA-mA-mG-mU-mC-S-mG-S-mG (5′->3′; SEQ ID NO: 27); or
f) the sense strand of the dsRNAi oligonucleotide consists of the sequence and all of the modifications of mU-S-mG-mU-mU-mU-mG-mU-fC-fA-fG-fC-mA-mA-mA-mG-mA-mU-mG-mU-mA-mG-mC-mA-mG-mC-mC-mG-[ademA-GalNAc]-[ademA-GalNAc]-[ademA-GalNAc]-mG-mG-mC-mU-mG-mC (5′->3′; SEQ ID NO: 23), and the antisense strand of the dsRNAi oligonucleotide for reducing KHK expression consists of the sequence and all of the modifications of [MePhosphonate-4O-mU]-S-fA-S-fC-fA-fU-mC-fU-mU-mU-fG-mC-mU-mG-fA-mC-mA-mA-mA-mC-mA-S-mG-S-mG (5′->3′; SEQ ID NO: 29);
wherein mC, mA, mG, mU=2′-OMe ribonucleosides; fA, fC, fG, fU=2′-F ribonucleosides; “-”=phosphodiester linkage, “—S—”=phosphorothioate linkage, wherein [ademA-GalNAc] designates 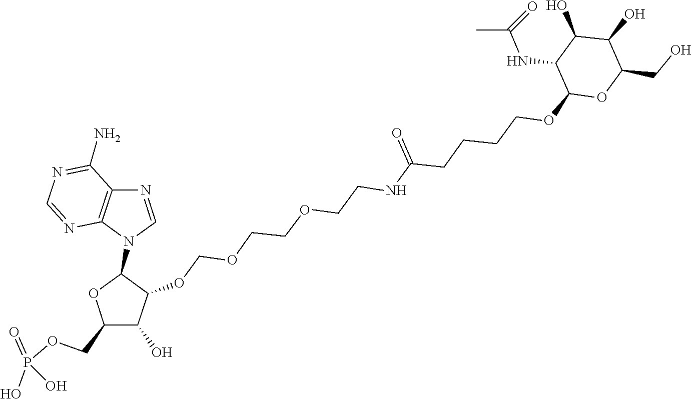 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]
and wherein [MePhosphonate-4O-mU]—S— designates

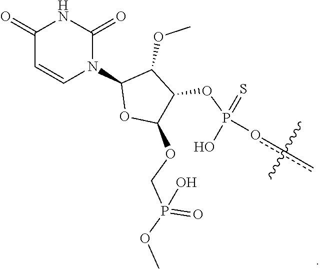

5. The method of claim 1, wherein
a) the dsRNAi oligonucleotide comprises a sense strand according to SEQ ID NO: 22 and an antisense strand according to SEQ ID NO: 28, wherein the dsRNAi oligonucleotide has the structure: 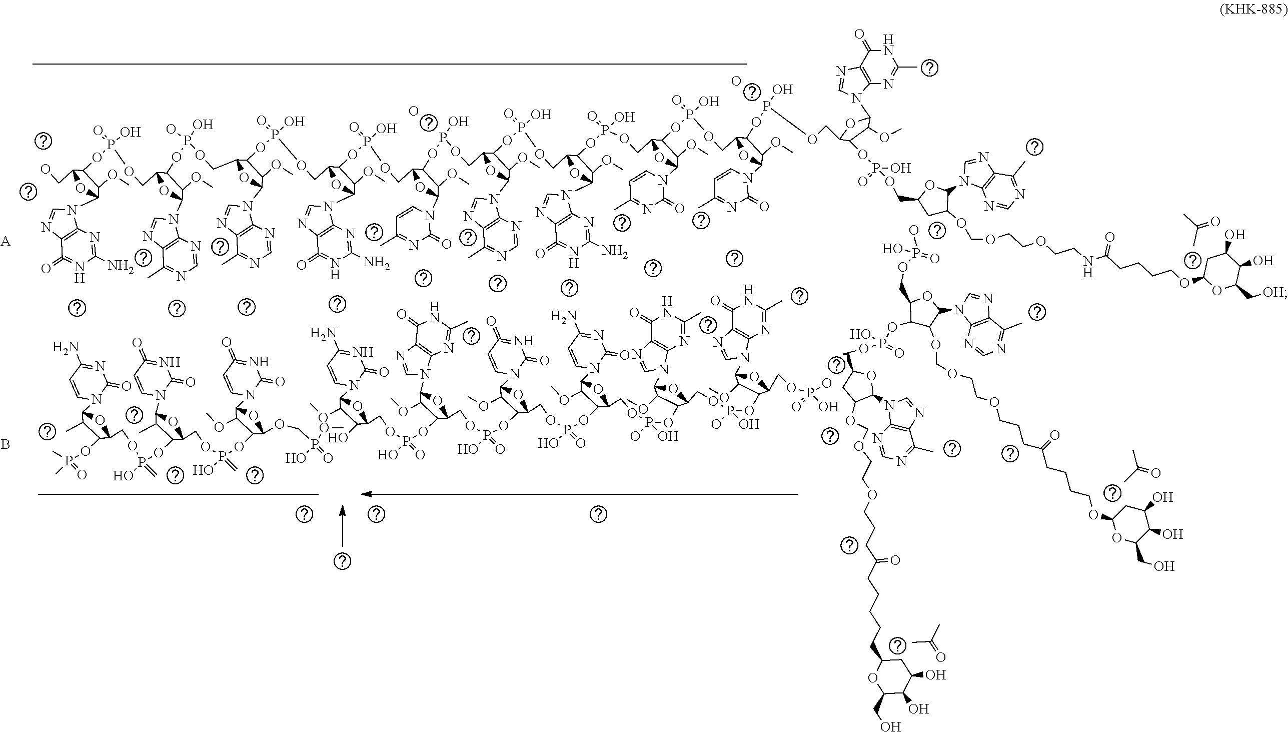 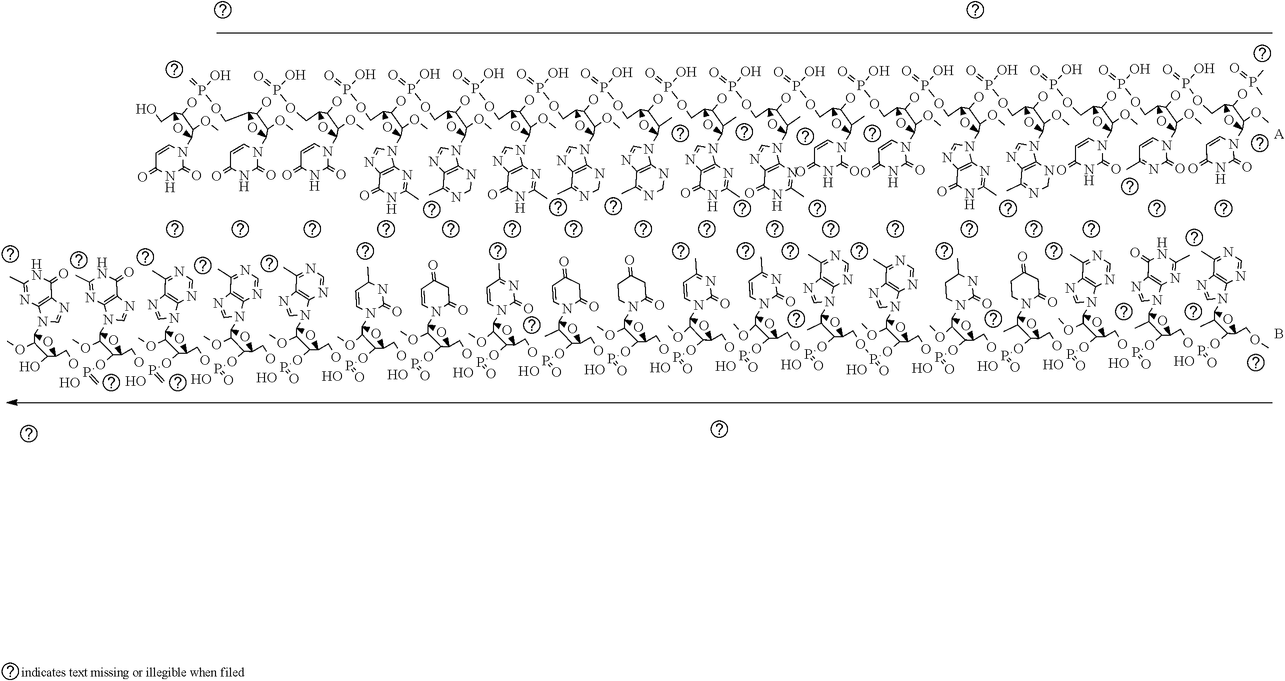
or
b) the dsRNAi oligonucleotide comprises a sense strand according to SEQ ID NO: 24 and an antisense strand according to SEQ ID NO: 30, wherein the dsRNAi oligonucleotide has the structure: 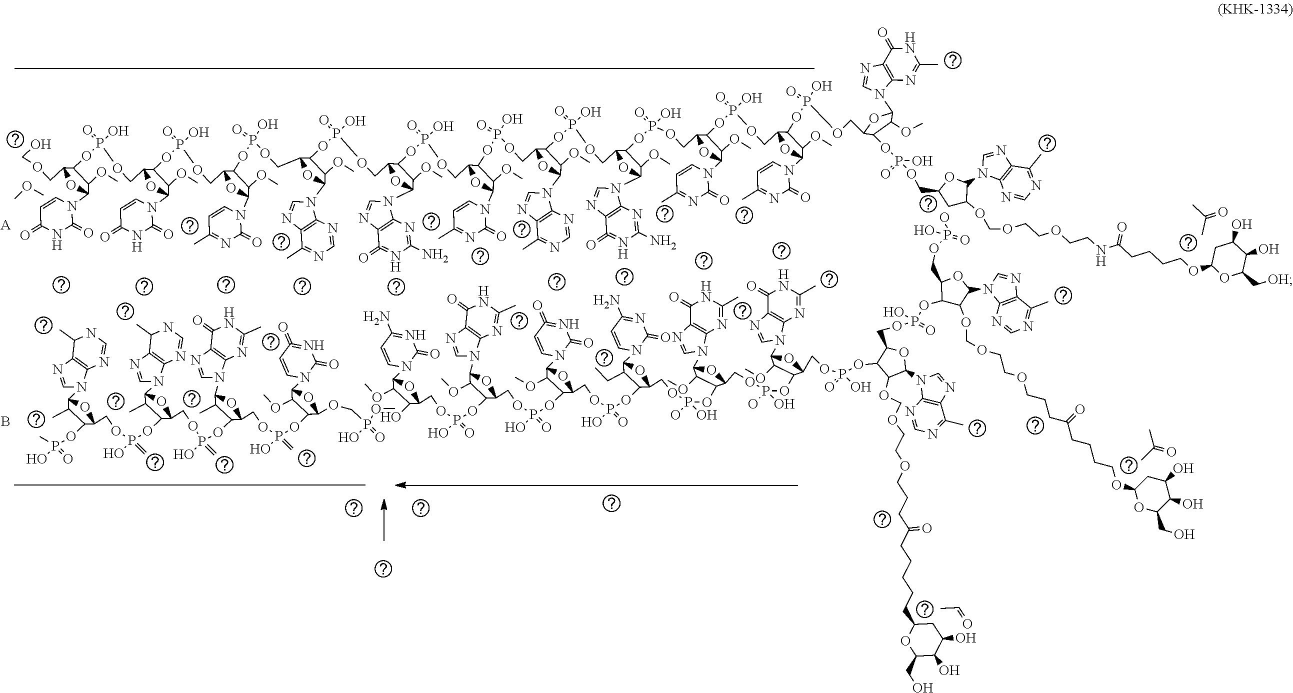 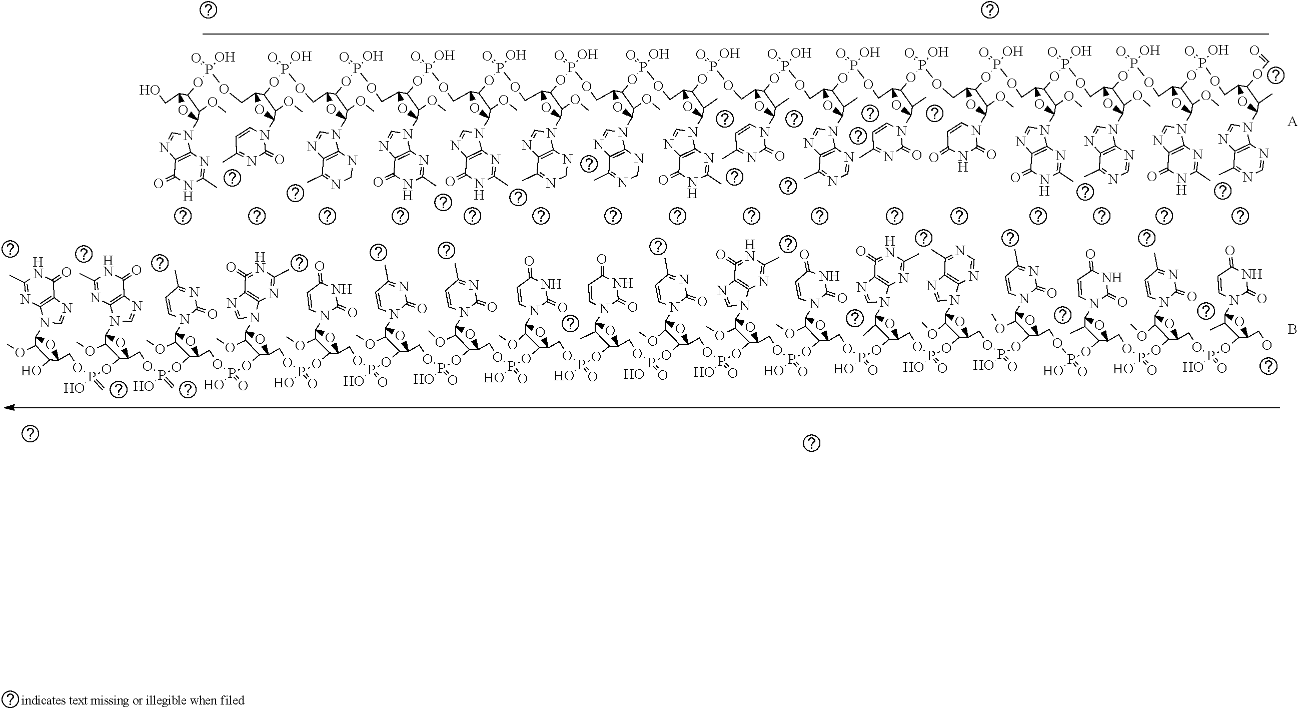
or
c) the dsRNAi oligonucleotide comprises a sense strand according to SEQ ID NO: 19 and an antisense strand according to SEQ ID NO: 25, wherein the dsRNAi oligonucleotide has the structure: 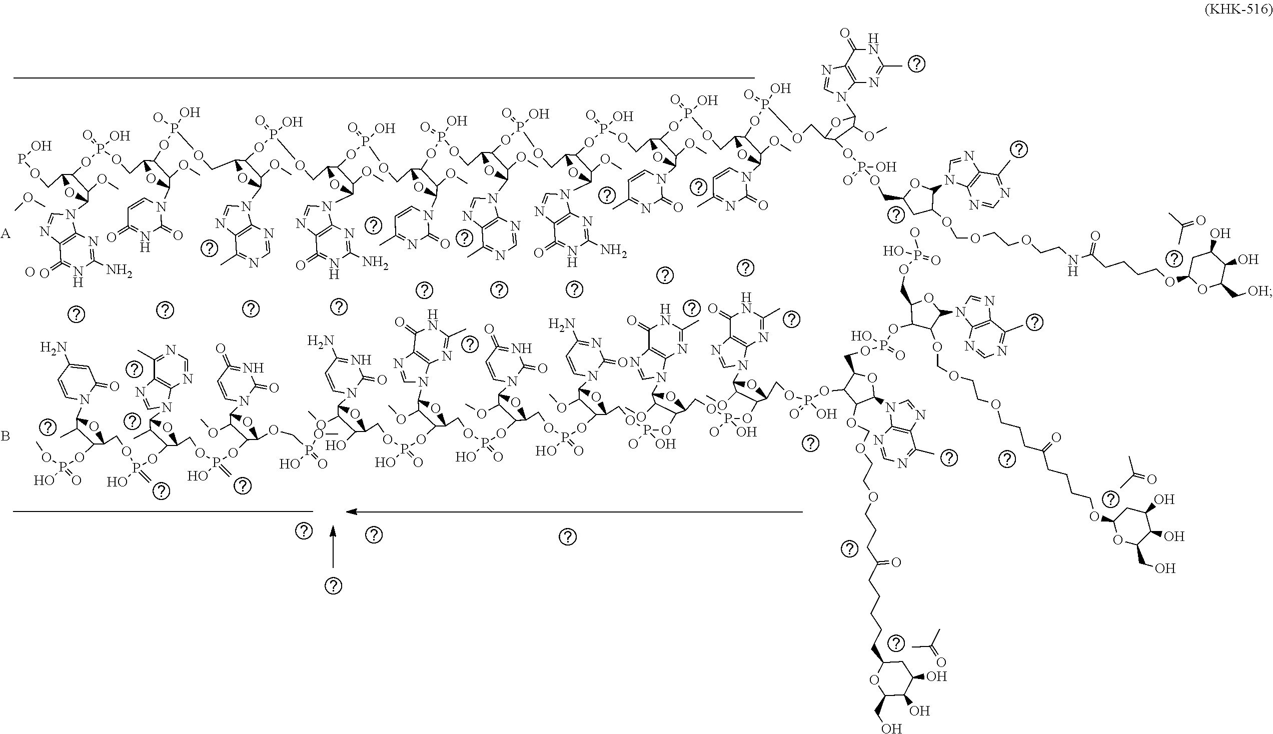 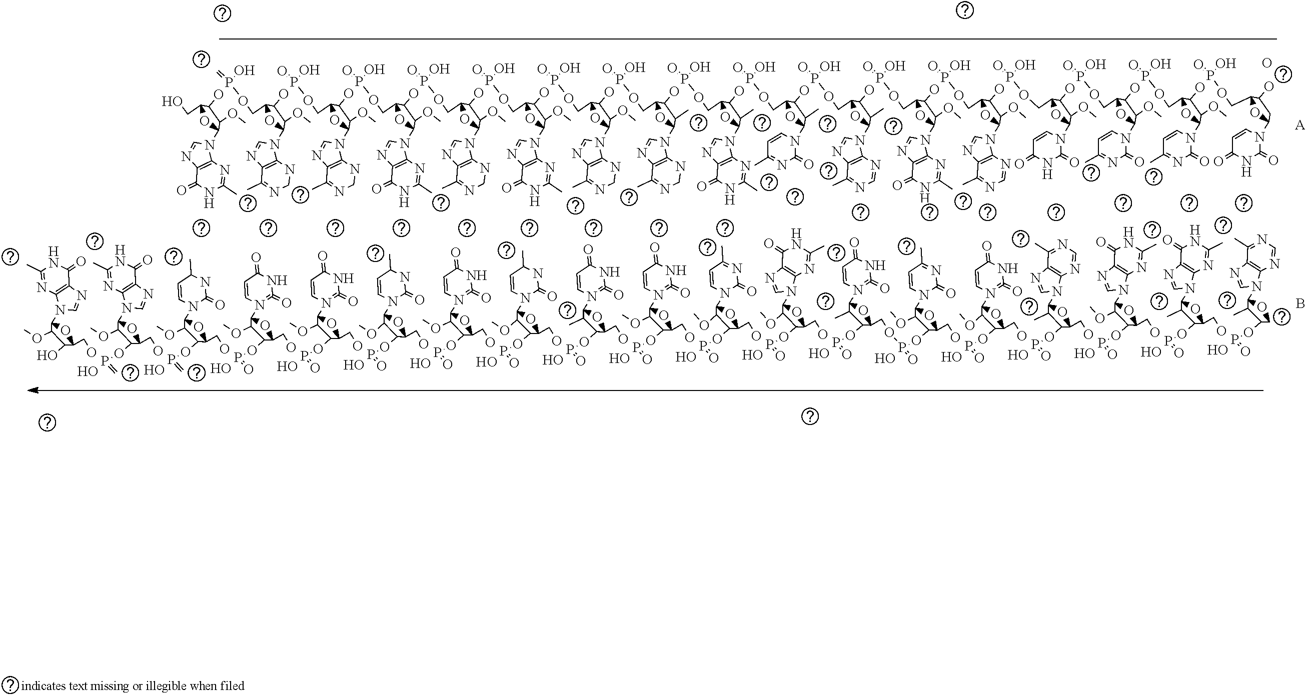
or
d) the dsRNAi oligonucleotide comprises a sense strand according to SEQ ID NO: 20 and an antisense strand according to SEQ ID NO: 26, wherein the dsRNAi oligonucleotide has the structure: 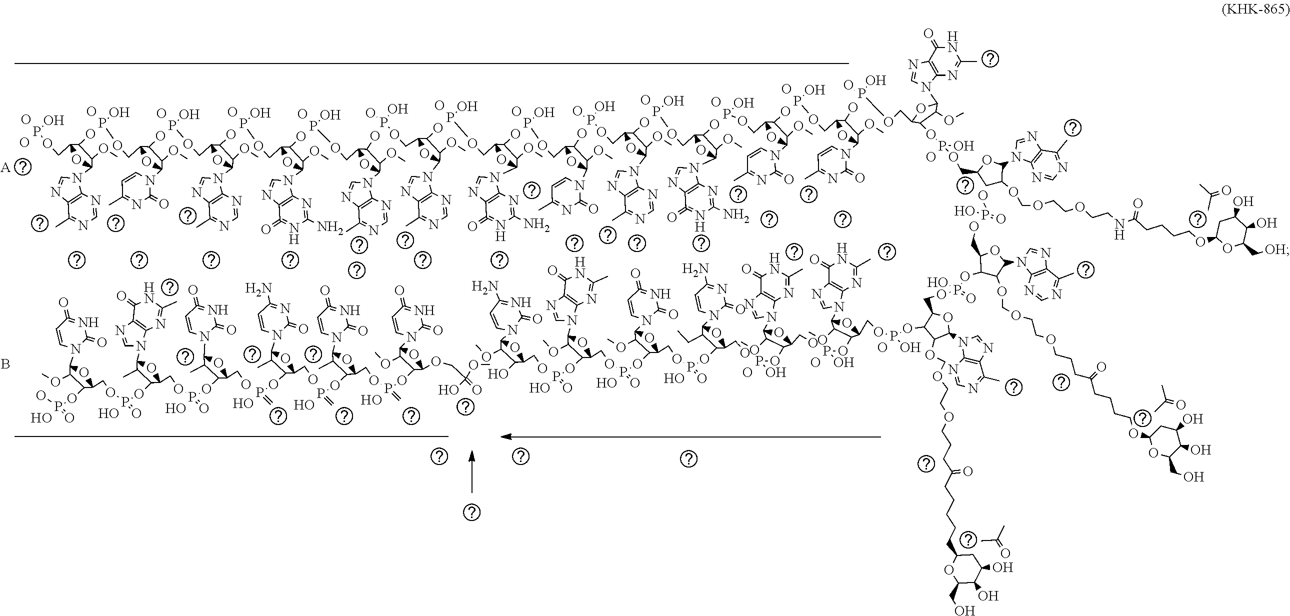 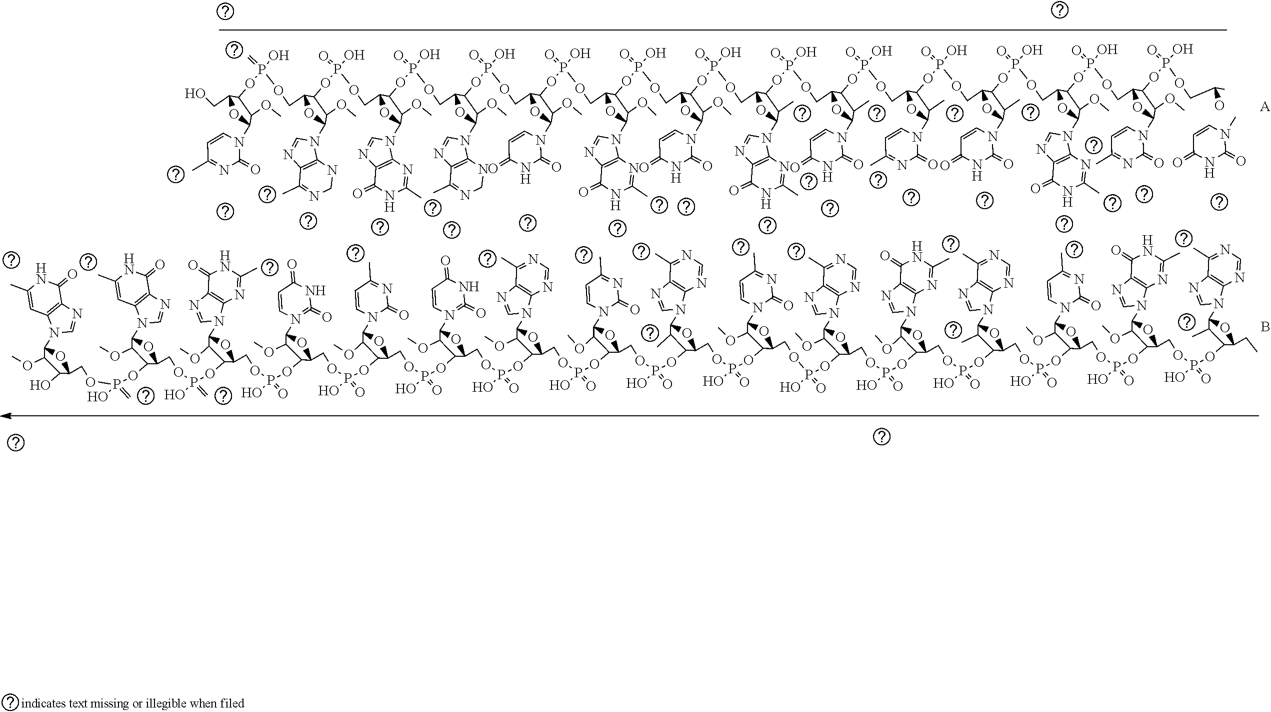
or
e) the dsRNAi oligonucleotide comprises a sense strand according to SEQ ID NO: 21 and an antisense strand according to SEQ ID NO: 27, wherein the dsRNAi oligonucleotide has the structure: 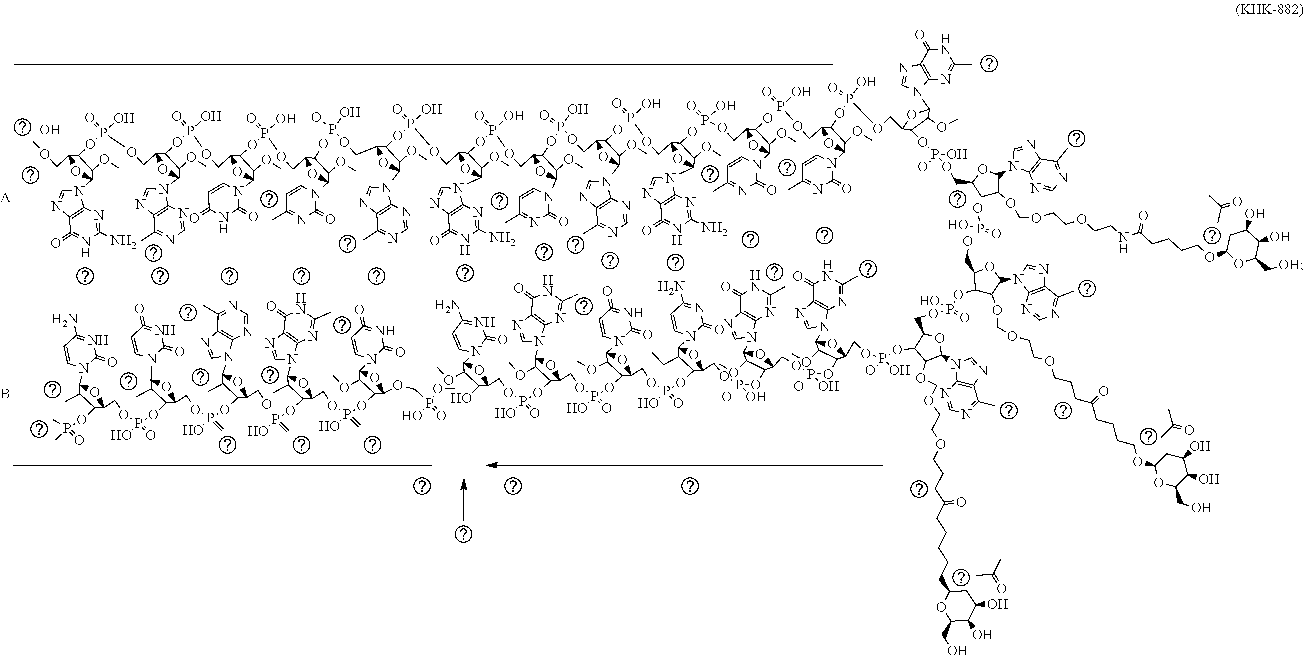 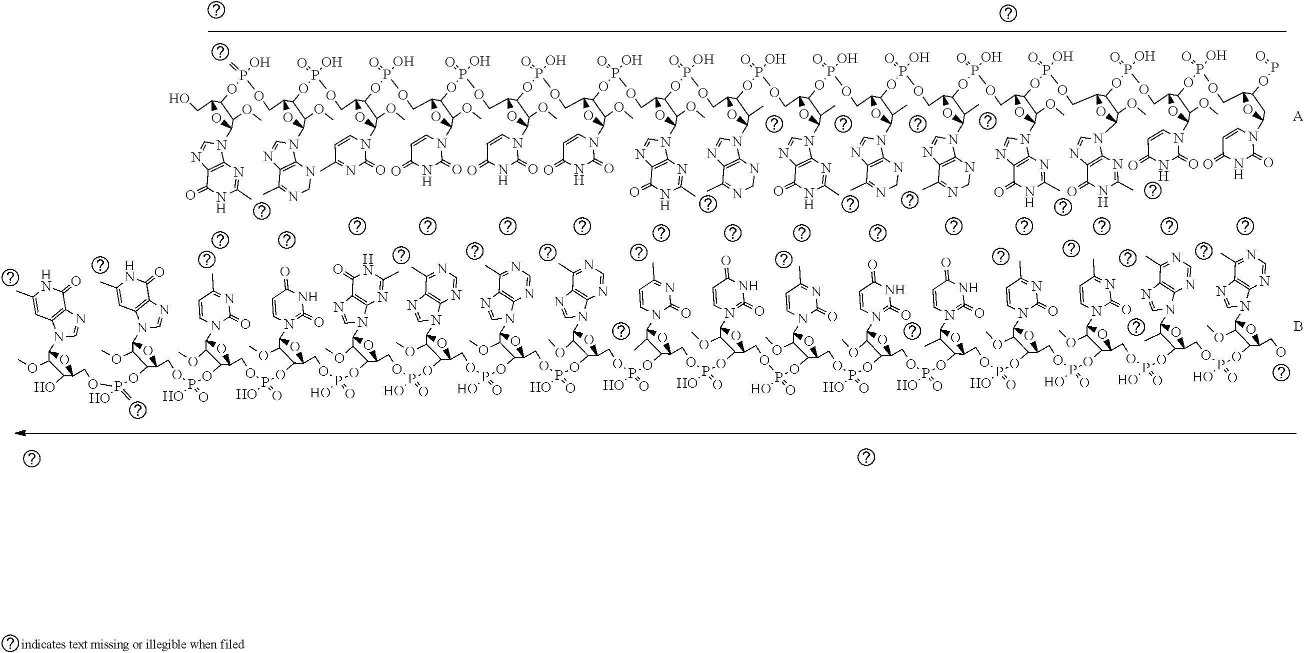
or
f) the dsRNAi oligonucleotide comprises a sense strand according to SEQ ID NO: 23 and an antisense strand according to SEQ ID NO: 29, wherein the dsRNAi oligonucleotide has the structure:

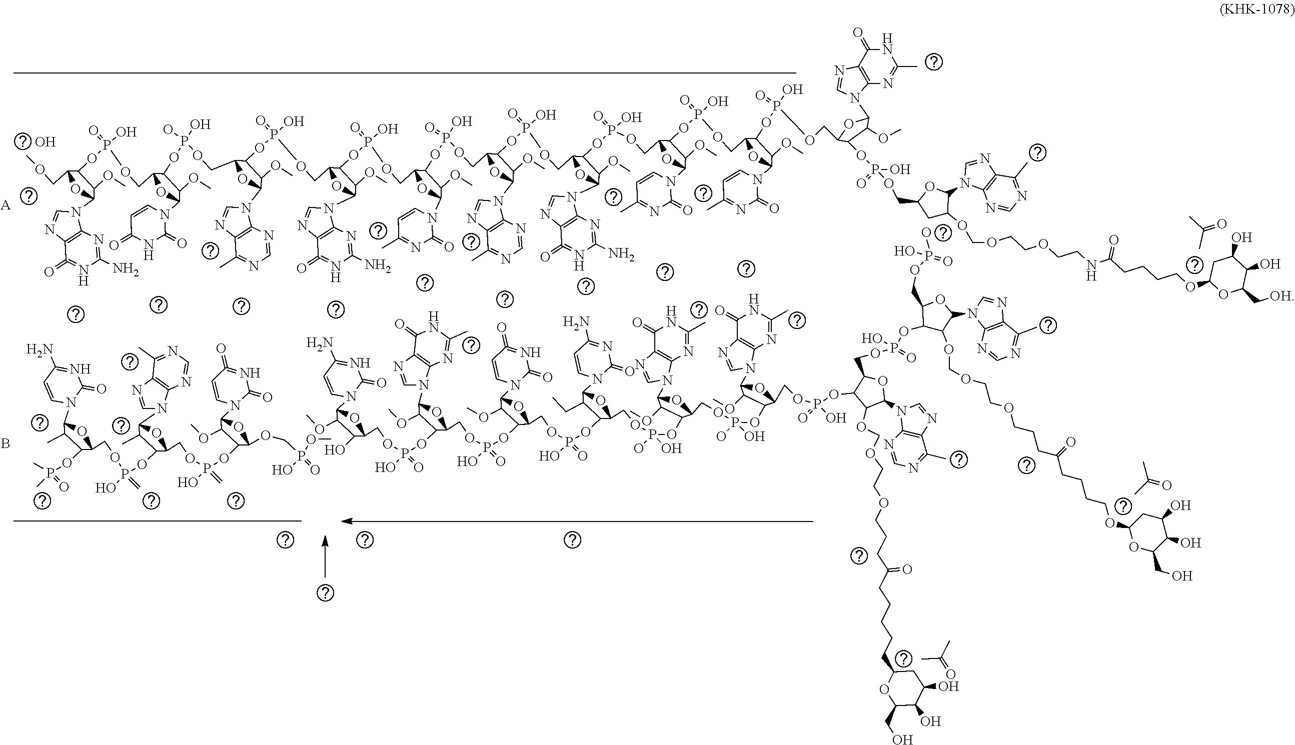

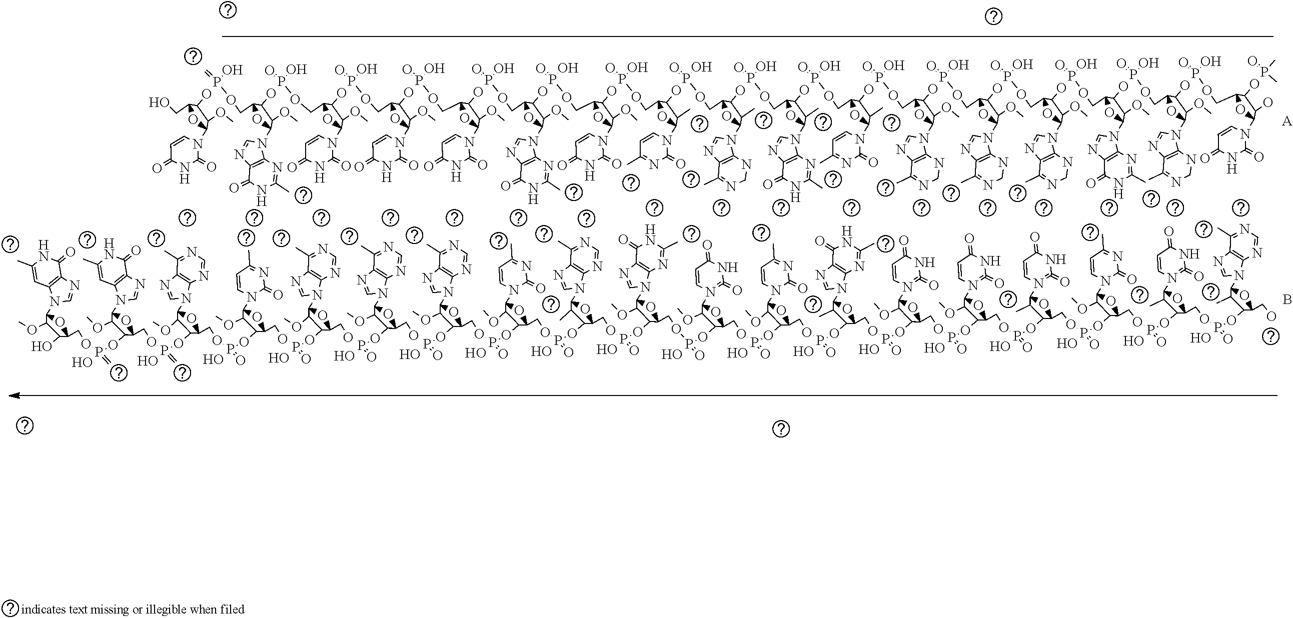

6. The method of claim 1, wherein the dsRNAi oligonucleotide reduces human KHK-C expression in the liver.

7. The method of claim 3, wherein the patient's NASH is in the compensated and/or decompensated cirrhotic stage.

8. The method of claim 1, wherein the patient is a human patient for whom weight loss is undesired.

9. The method of claim 1, wherein the dsRNAi oligonucleotide is administered subcutaneously in intervals of 1, 2, or 3 months at a dose below 100 mg.

10. The method of claim 1, wherein the dsRNAi oligonucleotide is administered in combination with at least one further agent that causes a loss of body weight and/or liver weight.

11. The method of claim 1, wherein the dsRNAi or pharmaceutically acceptable salt thereof is present as part of a pharmaceutical composition.

12. The method of claim 11, wherein the pharmaceutical composition is an aqueous solution for subcutaneous injection.

13. The method of claim 12, wherein the injection volume is not more than about 2 mL.

14. The method of claim 13, wherein the concentration of the dsRNAi or pharmaceutically acceptable salt thereof in the aqueous solution in the range from about 10 mg/mL to about 500 mg/mL.

15. The method of claim 14, wherein the pH value and the osmolality of the aqueous solution are in the physiologically acceptable range for a pharmaceutically acceptable composition.


SEQUENCE LISTING (34 sequences)

SEQ ID NO: 1 - RNA, 19 nt, synthetic construct
  source 1..19: mol_type = other RNA
gaagagaagcagatcctgt

SEQ ID NO: 2 - RNA, 19 nt, synthetic construct
  source 1..19: mol_type = other RNA
cagatgtgtctgctacaga

SEQ ID NO: 3 - RNA, 19 nt, synthetic construct
  source 1..19: mol_type = other RNA
gactttgagaaggttgatc

SEQ ID NO: 4 - RNA, 19 nt, synthetic construct
  source 1..19: mol_type = other RNA
tttgagaaggttgatctga

SEQ ID NO: 5 - RNA, 19 nt, synthetic construct
  source 1..19: mol_type = other RNA
tgtttgtcagcaaagatgt

SEQ ID NO: 6 - RNA, 19 nt, synthetic construct
  source 1..19: mol_type = other RNA
gcaggaagcactgagattc

SEQ ID NO: 7 - RNA, 36 nt, synthetic construct
  source 1..36: mol_type = other RNA
gaagagaagcagatcctgtagcagccgaaaggctgc

SEQ ID NO: 8 - RNA, 36 nt, synthetic construct
  source 1..36: mol_type = other RNA
cagatgtgtctgctacagaagcagccgaaaggctgc

SEQ ID NO: 9 - RNA, 36 nt, synthetic construct
  source 1..36: mol_type = other RNA
gactttgagaaggttgatcagcagccgaaaggctgc

SEQ ID NO: 10 - RNA, 36 nt, synthetic construct
  source 1..36: mol_type = other RNA
tttgagaaggttgatctgaagcagccgaaaggctgc

SEQ ID NO: 11 - RNA, 36 nt, synthetic construct
  source 1..36: mol_type = other RNA
tgtttgtcagcaaagatgtagcagccgaaaggctgc

SEQ ID NO: 12 - RNA, 36 nt, synthetic construct
  source 1..36: mol_type = other RNA
gcaggaagcactgagattcagcagccgaaaggctgc

SEQ ID NO: 13 - RNA, 22 nt, synthetic construct
  source 1..22: mol_type = other RNA
tacaggatctgcttctcttcgg

SEQ ID NO: 14 - RNA, 22 nt, synthetic construct
  source 1..22: mol_type = other RNA
ttctgtagcagacacatctggg

SEQ ID NO: 15 - RNA, 22 nt, synthetic construct
  source 1..22: mol_type = other RNA
tgatcaaccttctcaaagtcgg

SEQ ID NO: 16 - RNA, 22 nt, synthetic construct
  source 1..22: mol_type = other RNA
ttcagatcaaccttctcaaagg

SEQ ID NO: 17 - RNA, 22 nt, synthetic construct
  source 1..22: mol_type = other RNA
tacatctttgctgacaaacagg

SEQ ID NO: 18 - RNA, 22 nt, synthetic construct
  source 1..22: mol_type = other RNA
tgaatctcagtgcttcctgcgg

SEQ ID NO: 19 - RNA, 36 nt, synthetic construct
  source 1..36: mol_type = other RNA
  modified_base 1: mod_base = OTHER; note = 2'-O-methylguanosine with a phosphorothioate linkage
  modified_base 2..3: mod_base = OTHER; note = 2'-O-methyladenosine
  modified_base 4: mod_base = gm
  modified_base 5: mod_base = OTHER; note = 2'-O-methyladenosine
  modified_base 6: mod_base = gm
  modified_base 7: mod_base = OTHER; note = 2'-O-methyladenosine
  modified_base 8: mod_base = OTHER; note = 2'-fluoroadenosine
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluoroadenosine
  modified_base 12: mod_base = gm
  modified_base 13: mod_base = OTHER; note = 2'-O-methyladenosine
  modified_base 14: mod_base = um
  modified_base 15..16: mod_base = cm
  modified_base 17: mod_base = um
  modified_base 18: mod_base = gm
  modified_base 19: mod_base = um
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25..26: mod_base = cm
  modified_base 27: mod_base = gm
  modified_base 28..30: mod_base = OTHER; note = 2'-aminodiethoxymethanol-Adenine-GalNAc
  modified_base 31..32: mod_base = gm
  modified_base 33: mod_base = cm
  modified_base 34: mod_base = um
  modified_base 35: mod_base = gm
  modified_base 36: mod_base = cm
naagagannnngatcctgtagcagccgnnnggctgc

SEQ ID NO: 20 - RNA, 36 nt, synthetic construct
  source 1..36: mol_type = other RNA
  modified_base 1: mod_base = OTHER; note = 2'-O-methylcytidine with a phosphorothioate linkage
  modified_base 2: mod_base = OTHER; note = 2'-O-methyladenosine
  modified_base 3: mod_base = gm
  modified_base 4: mod_base = OTHER; note = 2'-O-methyladenosine
  modified_base 5: mod_base = um
  modified_base 6: mod_base = gm
  modified_base 7: mod_base = um
  modified_base 8: mod_base = OTHER; note = 2'-fluoroguanosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = gm
  modified_base 13: mod_base = cm
  modified_base 14: mod_base = um
  modified_base 15: mod_base = OTHER; note = 2'-O-methyladenosine
  modified_base 16: mod_base = cm
  modified_base 17: mod_base = OTHER; note = 2'-O-methyladenosine
  modified_base 18: mod_base = gm
  modified_base 19..20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25..26: mod_base = cm
  modified_base 27: mod_base = gm
  modified_base 28..30: mod_base = OTHER; note = 2'-aminodiethoxymethanol-Adenine-GalNAc
  modified_base 31..32: mod_base = gm
  modified_base 33: mod_base = cm
  modified_base 34: mod_base = um
  modified_base 35: mod_base = gm
  modified_base 36: mod_base = cm
nagatgtnnnngctacagaagcagccgnnnggctgc

SEQ ID NO: 21 - RNA, 36 nt, synthetic construct
  source 1..36: mol_type = other RNA
  modified_base 1: mod_base = OTHER; note = 2'-O-methylguanosine with a phosphorothioate linkage
  modified_base 2: mod_base = OTHER; note = 2'-O-methyladenosine
  modified_base 3: mod_base = cm
  modified_base 4..6: mod_base = um
  modified_base 7: mod_base = gm
  modified_base 8: mod_base = OTHER; note = 2'-fluoroadenosine
  modified_base 9: mod_base = OTHER; note = 2'-fluoroguanosine
  modified_base 10..11: mod_base = OTHER; note = 2'-fluoroadenosine
  modified_base 12..13: mod_base = gm
  modified_base 14..15: mod_base = um
  modified_base 16: mod_base = gm
  modified_base 17: mod_base = OTHER; note = 2'-O-methyladenosine
  modified_base 18: mod_base = um
  modified_base 19: mod_base = cm
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25..26: mod_base = cm
  modified_base 27: mod_base = gm
  modified_base 28..30: mod_base = OTHER; note = 2'-aminodiethoxymethanol-Adenine-GalNAc
  modified_base 31..32: mod_base = gm
  modified_base 33: mod_base = cm
  modified_base 34: mod_base = um
  modified_base 35: mod_base = gm
  modified_base 36: mod_base = cm
nactttgnnnnggttgatcagcagccgnnnggctgc

SEQ ID NO: 22 - RNA, 36 nt, synthetic construct
  source 1..36: mol_type = other RNA
  modified_base 1: mod_base = OTHER; note = 2'-O-methyluridine with a phosphorothioate linkage
  modified_base 2..3: mod_base = um
  modified_base 4: mod_base = gm
  modified_base 5: mod_base = OTHER; note = 2'-O-methyladenosine
  modified_base 6: mod_base = gm
  modified_base 7: mod_base = OTHER; note = 2'-O-methyladenosine
  modified_base 8: mod_base = OTHER; note = 2'-fluoroadenosine
  modified_base 9..10: mod_base = OTHER; note = 2'-fluoroguanosine
  modified_base 11: mod_base = OTHER; note = 2'-fluorouridine
  modified_base 12: mod_base = um
  modified_base 13: mod_base = gm
  modified_base 14: mod_base = OTHER; note = 2'-O-methyladenosine
  modified_base 15: mod_base = um
  modified_base 16: mod_base = cm
  modified_base 17: mod_base = um
  modified_base 18: mod_base = gm
  modified_base 19..20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25..26: mod_base = cm
  modified_base 27: mod_base = gm
  modified_base 28..30: mod_base = OTHER; note = 2'-aminodiethoxymethanol-Adenine-GalNAc
  modified_base 31..32: mod_base = gm
  modified_base 33: mod_base = cm
  modified_base 34: mod_base = um
  modified_base 35: mod_base = gm
  modified_base 36: mod_base = cm
nttgagannnntgatctgaagcagccgnnnggctgc

SEQ ID NO: 23 - RNA, 36 nt, synthetic construct
  source 1..36: mol_type = other RNA
  modified_base 1: mod_base = OTHER; note = 2'-O-methyluridine with a phosphorothioate linkage
  modified_base 2: mod_base = gm
  modified_base 3..5: mod_base = um
  modified_base 6: mod_base = gm
  modified_base 7: mod_base = um
  modified_base 8: mod_base = OTHER; note = 2'-fluorocytidine
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluorocytidine
  modified_base 12..14: mod_base = OTHER; note = 2'-O-methyladenosine
  modified_base 15: mod_base = gm
  modified_base 16: mod_base = OTHER; note = 2'-O-methyladenosine
  modified_base 17: mod_base = um
  modified_base 18: mod_base = gm
  modified_base 19: mod_base = um
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25..26: mod_base = cm
  modified_base 27: mod_base = gm
  modified_base 28..30: mod_base = OTHER; note = 2'-aminodiethoxymethanol-Adenine-GalNAc
  modified_base 31..32: mod_base = gm
  modified_base 33: mod_base = cm
  modified_base 34: mod_base = um
  modified_base 35: mod_base = gm
  modified_base 36: mod_base = cm
ngtttgtnnnnaaagatgtagcagccgnnnggctgc

SEQ ID NO: 24 - RNA, 36 nt, synthetic construct
  source 1..36: mol_type = other RNA
  modified_base 1: mod_base = OTHER; note = 2'-O-methylguanosine with a phosphorothioate linkage
  modified_base 2: mod_base = cm
  modified_base 3: mod_base = OTHER; note = 2'-O-methyladenosine
  modified_base 4..5: mod_base = gm
  modified_base 6..7: mod_base = OTHER; note = 2'-O-methyladenosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = gm
  modified_base 14: mod_base = OTHER; note = 2'-O-methyladenosine
  modified_base 15: mod_base = gm
  modified_base 16: mod_base = OTHER; note = 2'-O-methyladenosine
  modified_base 17..18: mod_base = um
  modified_base 19: mod_base = cm
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25..26: mod_base = cm
  modified_base 27: mod_base = gm
  modified_base 28..30: mod_base = OTHER; note = 2'-aminodiethoxymethanol-Adenine-GalNAc
  modified_base 31..32: mod_base = gm
  modified_base 33: mod_base = cm
  modified_base 34: mod_base = um
  modified_base 35: mod_base = gm
  modified_base 36: mod_base = cm
ncaggaannnntgagattcagcagccgnnnggctgc

SEQ ID NO: 25 - RNA, 22 nt, synthetic construct
  source 1..22: mol_type = other RNA
  modified_base 1: mod_base = OTHER; note = 5'-methoxyphosphonate-4'-oxy modified 2'-O-methyl uridine with a phosphorothioate linkage
  modified_base 2: mod_base = OTHER; note = 2'-fluoroadenosine with a phosphorothioate linkage
  modified_base 3: mod_base = OTHER; note = 2'-fluorocytidine
  modified_base 4: mod_base = OTHER; note = 2'-fluoroadenosine
  modified_base 5: mod_base = OTHER; note = 2'-fluoroguanosine
  modified_base 6: mod_base = gm
  modified_base 7: mod_base = OTHER; note = 2'-fluoroadenosine
  modified_base 8: mod_base = um
  modified_base 9: mod_base = cm
  modified_base 10: mod_base = OTHER; note = 2'-fluorouridine
  modified_base 11: mod_base = gm
  modified_base 12: mod_base = cm
  modified_base 13: mod_base = um
  modified_base 14: mod_base = OTHER; note = 2'-fluorouridine
  modified_base 15: mod_base = cm
  modified_base 16: mod_base = um
  modified_base 17: mod_base = cm
  modified_base 18..19: mod_base = um
  modified_base 20: mod_base = OTHER; note = 2'-O-methylcytidine with a phosphorothioate linkage
  modified_base 21: mod_base = OTHER; note = 2'-O-methylguanosine with a phosphorothioate linkage
  modified_base 22: mod_base = gm
nnnnngntcngctnctcttnng

SEQ ID NO: 26 - RNA, 22 nt, synthetic construct
  source 1..22: mol_type = other RNA
  modified_base 1: mod_base = OTHER; note = 5'-methoxyphosphonate-4'-oxy modified 2'-O-methyl uridine with a phosphorothioate linkage
  modified_base 2: mod_base = OTHER; note = 2'-fluorouridine with a phosphorothioate linkage
  modified_base 3: mod_base = OTHER; note = 2'-fluorocytidine with a phosphorothioate linkage
  modified_base 4: mod_base = OTHER; note = 2'-fluorouridine
  modified_base 5: mod_base = OTHER; note = 2'-fluoroguanosine
  modified_base 6: mod_base = um
  modified_base 7: mod_base = OTHER; note = 2'-fluoroadenosine
  modified_base 8: mod_base = gm
  modified_base 9: mod_base = cm
  modified_base 10: mod_base = OTHER; note = 2'-fluoroadenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-O-methyladenosine
  modified_base 17: mod_base = um
  modified_base 18: mod_base = cm
  modified_base 19: mod_base = um
  modified_base 20..21: mod_base = OTHER; note = 2'-O-methylguanosine with a phosphorothioate linkage
  modified_base 22: mod_base = gm
nnnnntngcngacncatctnng

SEQ ID NO: 27 - RNA, 22 nt, synthetic construct
  source 1..22: mol_type = other RNA
  modified_base 1: mod_base = OTHER; note = 5'-methoxyphosphonate-4'-oxy modified 2'-O-methyl uridine with a phosphorothioate linkage
  modified_base 2: mod_base = OTHER; note = 2'-fluoroguanosine with a phosphorothioate linkage
  modified_base 3: mod_base = OTHER; note = 2'-fluoroadenosine with a phosphorothioate linkage
  modified_base 4: mod_base = OTHER; note = 2'-fluorouridine
  modified_base 5: mod_base = OTHER; note = 2'-fluorocytidine
  modified_base 6: mod_base = OTHER; note = 2'-O-methyladenosine
  modified_base 7: mod_base = OTHER; note = 2'-fluoroadenosine
  modified_base 8..9: mod_base = cm
  modified_base 10: mod_base = OTHER; note = 2'-fluorouridine
  modified_base 11: mod_base = um
  modified_base 12: mod_base = cm
  modified_base 13: mod_base = um
  modified_base 14: mod_base = OTHER; note = 2'-fluorocytidine
  modified_base 15..17: mod_base = OTHER; note = 2'-O-methyladenosine
  modified_base 18: mod_base = gm
  modified_base 19: mod_base = um
  modified_base 20: mod_base = OTHER; note = 2'-O-methylcytidine with a phosphorothioate linkage
  modified_base 21: mod_base = OTHER; note = 2'-O-methylguanosine with a phosphorothioate linkage
  modified_base 22: mod_base = gm
nnnnnanccntctnaaagtnng

SEQ ID NO: 28 - RNA, 22 nt, synthetic construct
  source 1..22: mol_type = other RNA
  modified_base 1: mod_base = OTHER; note = 5'-methoxyphosphonate-4'-oxy modified 2'-O-methyl uridine with a phosphorothioate linkage
  modified_base 2: mod_base = OTHER; note = 2'-fluorouridine with a phosphorothioate linkage
  modified_base 3: mod_base = OTHER; note = 2'-fluorocytidine with a phosphorothioate linkage
  modified_base 4: mod_base = OTHER; note = 2'-fluoroadenosine
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-fluorouridine
  modified_base 8: mod_base = cm
  modified_base 9: mod_base = OTHER; note = 2'-O-methyladenosine
  modified_base 10: mod_base = OTHER; note = 2'-fluoroadenosine
  modified_base 11..12: mod_base = cm
  modified_base 13: mod_base = um
  modified_base 14: mod_base = OTHER; note = 2'-fluorouridine
  modified_base 15: mod_base = cm
  modified_base 16: mod_base = um
  modified_base 17: mod_base = cm
  modified_base 18..19: mod_base = OTHER; note = 2'-O-methyladenosine
  modified_base 20: mod_base = OTHER; note = 2'-O-methyladenosine with a phosphorothioate linkage
  modified_base 21: mod_base = OTHER; note = 2'-O-methylguanosine with a phosphorothioate linkage
  modified_base 22: mod_base = gm
nnnnnancancctnctcaanng

SEQ ID NO: 29 - RNA, 22 nt, synthetic construct
  source 1..22: mol_type = other RNA
  modified_base 1: mod_base = OTHER; note = 5'-methoxyphosphonate-4'-oxy modified 2'-O-methyl uridine with a phosphorothioate linkage
  modified_base 2: mod_base = OTHER; note = 2'-fluoroadenosine with a phosphorothioate linkage
  modified_base 3: mod_base = OTHER; note = 2'-fluorocytidine
  modified_base 4: mod_base = OTHER; note = 2'-fluoroadenosine
  modified_base 5: mod_base = OTHER; note = 2'-fluorouridine
  modified_base 6: mod_base = cm
  modified_base 7: mod_base = OTHER; note = 2'-fluorouridine
  modified_base 8..9: mod_base = um
  modified_base 10: mod_base = OTHER; note = 2'-fluoroguanosine
  modified_base 11: mod_base = cm
  modified_base 12: mod_base = um
  modified_base 13: mod_base = gm
  modified_base 14: mod_base = OTHER; note = 2'-fluoroadenosine
  modified_base 15: mod_base = cm
  modified_base 16..18: mod_base = OTHER; note = 2'-O-methyladenosine
  modified_base 19: mod_base = cm
  modified_base 20: mod_base = OTHER; note = 2'-O-methyladenosine with a phosphorothioate linkage
  modified_base 21: mod_base = OTHER; note = 2'-O-methylguanosine with a phosphorothioate linkage
  modified_base 22: mod_base = gm
nnnnncnttnctgncaaacnng

SEQ ID NO: 30 - RNA, 22 nt, synthetic construct
  source 1..22: mol_type = other RNA
  modified_base 1: mod_base = OTHER; note = 5'-methoxyphosphonate-4'-oxy modified 2'-O-methyl uridine with a phosphorothioate linkage
  modified_base 2: mod_base = OTHER; note = 2'-fluoroguanosine with a phosphorothioate linkage
  modified_base 3: mod_base = OTHER; note = 2'-fluoroadenosine with a phosphorothioate linkage
  modified_base 4: mod_base = OTHER; note = 2'-fluoroadenosine
  modified_base 5: mod_base = OTHER; note = 2'-fluorouridine
  modified_base 6: mod_base = cm
  modified_base 7: mod_base = OTHER; note = 2'-fluorouridine
  modified_base 8: mod_base = cm
  modified_base 9: mod_base = OTHER; note = 2'-O-methyladenosine
  modified_base 10: mod_base = OTHER; note = 2'-fluoroguanosine
  modified_base 11: mod_base = um
  modified_base 12: mod_base = gm
  modified_base 13: mod_base = cm
  modified_base 14: mod_base = OTHER; note = 2'-fluorouridine
  modified_base 15: mod_base = um
  modified_base 16..17: mod_base = cm
  modified_base 18: mod_base = um
  modified_base 19: mod_base = gm
  modified_base 20: mod_base = OTHER; note = 2'-O-methylcytidine with a phosphorothioate linkage
  modified_base 21: mod_base = OTHER; note = 2'-O-methylguanosine with a phosphorothioate linkage
  modified_base 22: mod_base = gm
nnnnncncantgcntcctgnng

SEQ ID NO: 31 - DNA, 19 bp, synthetic construct
  source 1..19: mol_type = other DNA
tgccttcatgggctcaatg

SEQ ID NO: 32 - DNA, 18 bp, synthetic construct
  source 1..18: mol_type = other DNA
tcggccaccaggaagtca

SEQ ID NO: 33 - DNA, 14 bp, synthetic construct
  source 1..14: mol_type = other DNA
ccctggccatgttg

SEQ ID NO: 34 - RNA, 36 nt, synthetic construct
  source 1..36: mol_type = other RNA
  modified_base 1: mod_base = OTHER; note = 2'-O-methyladenosine with a phosphorothioate linkage
  modified_base 2: mod_base = gm
  modified_base 3: mod_base = OTHER; note = 2'-fluorouridine
  modified_base 4: mod_base = um
  modified_base 5: mod_base = gm
  modified_base 6..7: mod_base = um
  modified_base 8: mod_base = OTHER; note = 2'-fluorouridine
  modified_base 9: mod_base = OTHER; note = 2'-fluoroadenosine
  modified_base 10: mod_base = OTHER; note = 2'-fluoroguanosine
  modified_base 11: mod_base = cm
  modified_base 12: mod_base = OTHER; note = 2'-fluorouridine
  modified_base 13: mod_base = OTHER; note = 2'-fluoroadenosine
  modified_base 14: mod_base = um
  modified_base 15..16: mod_base = gm
  modified_base 17: mod_base = OTHER; note = 2'-fluorouridine
  modified_base 18: mod_base = gm
  modified_base 19..20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25..26: mod_base = cm
  modified_base 27: mod_base = gm
  modified_base 28..30: mod_base = OTHER; note = 2' aminodiethoxymethanol-Adenine-GalNAc
  modified_base 31..32: mod_base = gm
  modified_base 33: mod_base = cm
  modified_base 34: mod_base = um
  modified_base 35: mod_base = gm
  modified_base 36: mod_base = cm
ngntgttnnncnntggngaagcagccgnnnggctgc